SEQUENCE LISTING (10 sequences)

SEQ ID NO: 1 - DNA, 1494 bp, Pseudomonas sp. HZN6
atggctaacgataagggtgatataagcaaggacggtgtatcgcgacgcaaatttcttggcggtgccgttattggtgctgctgctgctgcaggcgttggctctcagatcctatcactgtccgctacggcacagggggcggataaagaaagagttggtccgctgcaaagcaacgtagattacgatgccgtcgtgatcggaggtgggtttgcgggggtaacggctgcaagggagttgagccgatccggcttgaaaacattagtgcttgagggccggagtcgcctaggtggccgcacgtttacgtctaagctcgatggtgagaaggtggagcttggaggtacttgggtacactggacccagcctaatgtgtggactgaggtcatgcattatggattagaaatcgaggaaaccgtcggtctcgctagtcctgaaactgttatttgggttactgataatcaggtgaagcgagcgccggcggcagaggcgttcgaaatatttggcgccgcttgtactgaatattacaaagaagcgcacaacatctacccacgtcccttcgatcctttctttgcaaaaaaagcgctccaggagatggatgggttgtcagcttctgagtacttaaataaactgtccctaacccgcgagcaaaaagacatgatggattcatggcttagcggtaatggacataactacccagagacgatcgcttatagcgagattatgcgctggtttgcactcagcaacttcaacatgcccactatgttcgactcaattgccagatataaaattaaatcaggtaccgtgagtcttctggaggccatggttgctgaaagtgatatggaagttcagctttcaacgcctgtgctaaaagttaagcaagacagtcatagggtacttatcaccactgaagagggcacaattgcggcatcggcagttgtcatggcagtgcctttaaacacgatgggtgacgttgagtacagtccgcgtctctctgatgcaaagtcagaaattgcctcccaaggccatgcgggtaagggtgttaagggttacattcgtataaagcaagatgtaggtaacgtaatgacctatgcgcccgctagaaacgatgtaacgcctttcacttcggtgtttacggaccacgtcggtgagaacggtacattgcttatcgcattttccgccgatcctaaactggtagacattaacgatagcaaagcggtcgaaaaggccctgcatccccttcttccaggcgtggaggtaacgtccagctatggctacgactggaatctcgatcccttttctaagggcacttggtgcacttatcgtccgggtcagacaacccgctatttaaccgagctgcagaagcgcgaaggccggctcttcttcgccggttccgacatggctaatggctggcgtggttttatagacggcgcgatagagagcggtcgcgaggtcgggtatcaggttgctagctatctcaaggggaaaaatagcaatgcgtga

SEQ ID NO: 2 - DNA, 1664 bp, Pseudomonas sp. HZN6
ggatccagggaagttggtgatagtttgattcccaagtgcttttctaaaaatccataccatggctaatgataagggagatataagtaaagatggtgtgagtaggaggaagtttctcggtggtgctgtgataggtgctgctgctgctgcaggtgttggatcacaaattttaagtctctccgctactgcacagggtgctgataaggaaagagttggaccacttcaaagtaatgtggattatgatgcagttgtgattggaggtggatttgctggtgtgactgctgcaagagaattatctagatcaggtctcaaaacacttgttttggagggaagaagtaggttgggtggaaggactttcacatccaagttggatggagaaaaagttgagttaggtggaacttgggtgcattggacacaaccaaacgtttggactgaagtgatgcactatggtctcgagattgaagagacagttggacttgcttctccagagactgttatatgggtgacagataatcaggttaagagggctccagctgcagaagcatttgagattttcggtgctgcatgtactgaatattacaaagaggctcataacatctatccaaggcctttcgatccatttttcgctaagaaagcattacaagagatggatggactcagtgcttccgagtaccttaataagttatctctcactagagaacagaaagatatgatggattcttggctttctggtaatggtcacaactatccagaaacaatagcatactctgagatcatgagatggtttgctctttcaaatttcaacatgcctactatgttcgattcaatcgctaggtataagataaaaagtggtacagtttcccttttggaggctatggtggcagaatctgatatggaggttcaactttcaactccagttttgaaggtgaaacaggattctcatagagttcttatcactacagaagagggtactattgctgcatcagctgttgtgatggcagtgccattgaatacaatgggagatgttgaatactctcctaggttatcagatgctaagagtgagattgcatcccaaggtcacgctggaaagggagttaaaggatacatcagaattaagcaggatgttggaaatgtgatgacatacgctccagcaaggaacgatgttactccttttacatctgttttcactgatcatgtgggtgaaaatggtactcttctcatagcttttagtgcagatcctaaacttgtggatatcaacgattccaaggctgttgaaaaagcattgcacccacttttgcctggtgttgaagtgacttcttcatatggatacgattggaatcttgatccattttctaagggtacttggtgcacatatagacctggacaaactacaaggtaccttacagaattgcagaaaagagagggtaggcttttctttgcaggaagtgatatggctaacggttggagaggttttattgatggtgctattgaatccggtagggaggttggttatcaggttgcttcatatctcaagggaaagaatagtaacgcataaaatctaagatgttttatcttggttgatcattgtttaatactcctagatagatgggtattcatctatctttttaaaattaattgtcagtacgagtgtttctgagctcaagctt

SEQ ID NO: 3 - protein, 497 aa, Pseudomonas sp. HZN6
MANDKGDISKDGVSRRKFLGGAVIGAAAAAGVGSQILSLSATAQGADKERVGPLQSNVDYDAVVIGGGFAGVTAARELSRSGLKTLVLEGRSRLGGRTFTSKLDGEKVELGGTWVHWTQPNVWTEVMHYGLEIEETVGLASPETVIWVTDNQVKRAPAAEAFEIFGAACTEYYKEAHNIYPRPFDPFFAKKALQEMDGLSASEYLNKLSLTREQKDMMDSWLSGNGHNYPETIAYSEIMRWFALSNFNMPTMFDSIARYKIKSGTVSLLEAMVAESDMEVQLSTPVLKVKQDSHRVLITTEEGTIAASAVVMAVPLNTMGDVEYSPRLSDAKSEIASQGHAGKGVKGYIRIKQDVGNVMTYAPARNDVTPFTSVFTDHVGENGTLLIAFSADPKLVDINDSKAVEKALHPLLPGVEVTSSYGYDWNLDPFSKGTWCTYRPGQTTRYLTELQKREGRLFFAGSDMANGWRGFIDGAIESGREVGYQVASYLKGKNSNA

SEQ ID NO: 4 - DNA, 1746 bp, Nicotiana tabacum
gaagcagaaatacatacaacatgtttccgctcataattctgatcagcttttcacttgcttccttgtctgaaactgctactggagctgttacaaatctttcagcctgcttaatcaaccacaatgtccataacttctctatttaccccacaagtagaaattactttaacttgctccacttctcccttcaaaatcttcgctttgctgcacctttcatgccgaaaccaaccttcattatcctaccaagcagtaaggaggagctcgtgagcaccattttttgttgcagaaaagcatcttatgaaatcagagtaaggtgcggcggacacagttacgaaggaacttcttacgtttcctttgacgcttctccattcgtgatcgttgacttgatgaaattagacgacgtttcagtagatttggattctgaaacagcttgggctcagggcggcgcaacaattggccaaatttattatgccattgccaaggtaagtgacgttcatgcattttcagcaggttcgggaccaacagtaggatctggaggtcatatttcaggtggtggatttggacttttatctagaaaattcggacttgctgctgataatgtcgttgatgctcttcttattgatgctgatggacggttattagaccgaaaagccatgggcgaagacgtgttttgggcaatcagaggtggcggcggtggaaattggggcattgtttatgcctggaaaattcgattactcaaagtgcctaaaatcgtaacaacttgtatgatctataggcctggatccaaacaatacgtggctcaaatacttgagaaatggcaaatagttactccaaatttggtcgatgattttactctaggagtactgctgagacctgcagatctacccgcggatatgaaatatggtaatactactcctattgaaatatttccccaattcaatgcactttatttgggtccaaaaactgaagttctttccatatcgaatgagacatttccggagctaggcgttaagaatgatgagtgcaaggaaatgacttgggtagagtcagcacttttcttctccgaattagctgacgttaacgggaactcgactggtgatatctcccgtctgaaagaacgttacatggacggaaaaggttttttcaaaggcaaaacggactacgtgaagaagccagtttcaatggatgggatgctaacatttcttgtggaactcgagaaaaacccgaagggatatcttgtctttgatccttatggcggagccatggacaagattagtgatcaagctattgctttccctcatagaaaaggtaaccttttcgcgattcagtatctagcacagtggaatgaagaggacgattacatgagcgacgtttacatggagtggataagaggattttacaatacaatgacgccctttgtttcaagctcgccaaggggagcttatatcaactacttggatatggatcttggagtgaatatggtcgacgactacttattgcgaaatgctagtagcagtagtccttcttcctctgttgatgctgtggagagagctagagcgtggggtgagatgtatttcttgcataactatgataggttggttaaagctaagacacaaattgatccactaaatgtttttcgacatgaacagagtattcctcctatgcttggttcaacgcaagagcacaagtatagcagtgaatgagatttaaaatgtactaccttgagagagattccgttgttagttttcc

SEQ ID NO: 5 - protein, 559 aa, Nicotiana tabacum
MFPLIILISFSLASLSETATGAVTNLSACLINHNVHNFSIYPTSRNYFNLLHFSLQNLRFAAPFMPKPTFIILPSSKEELVSTIFCCRKASYEIRVRCGGHSYEGTSYVSFDASPFVIVDLMKLDDVSVDLDSETAWAQGGATIGQIYYAIAKVSDVHAFSAGSGPTVGSGGHISGGGFGLLSRKFGLAADNVVDALLIDADGRLLDRKAMGEDVFWAIRGGGGGNWGIVYAWKIRLLKVPKIVTTCMIYRPGSKQYVAQILEKWQIVTPNLVDDFTLGVLLRPADLPADMKYGNTTPIEIFPQFNALYLGPKTEVLSISNETFPELGVKNDECKEMTWVESALFFSELADVNGNSTGDISRLKERYMDGKGFFKGKTDYVKKPVSMDGMLTFLVELEKNPKGYLVFDPYGGAMDKISDQAIAFPHRKGNLFAIQYLAQWNEEDDYMSDVYMEWIRGFYNTMTPFVSSSPRGAYINYLDMDLGVNMVDDYLLRNASSSSPSSSVDAVERARAWGEMYFLHNYDRLVKAKTQIDPLNVFRHEQSIPPMLGSTQEHKYSSE

SEQ ID NO: 6 - DNA, 1785 bp, Artificial Sequence
  BBLa-PAO fusion construct
ggatccgaagcagaaatacatacaacatgtttccgctcataattctgatcagcttttcacttgcttccttgtctgaaactgctactggagctgttacaaatctttcagcctgcttaatcaaccacaatgtccataacttctctatttaccccacaagtagaaattactttaacttggccatggctaatgataagggagatataagtaaagatggtgtgagtaggaggaagtttctcggtggtgctgtgataggtgctgctgctgctgcaggtgttggatcacaaattttaagtctctccgctactgcacagggtgctgataaggaaagagttggaccacttcaaagtaatgtggattatgatgcagttgtgattggaggtggatttgctggtgtgactgctgcaagagaattatctagatcaggtctcaaaacacttgttttggagggaagaagtaggttgggtggaaggactttcacatccaagttggatggagaaaaagttgagttaggtggaacttgggtgcattggacacaaccaaacgtttggactgaagtgatgcactatggtctcgagattgaagagacagttggacttgcttctccagagactgttatatgggtgacagataatcaggttaagagggctccagctgcagaagcatttgagattttcggtgctgcatgtactgaatattacaaagaggctcataacatctatccaaggcctttcgatccatttttcgctaagaaagcattacaagagatggatggactcagtgcttccgagtaccttaataagttatctctcactagagaacagaaagatatgatggattcttggctttctggtaatggtcacaactatccagaaacaatagcatactctgagatcatgagatggtttgctctttcaaatttcaacatgcctactatgttcgattcaatcgctaggtataagataaaaagtggtacagtttcccttttggaggctatggtggcagaatctgatatggaggttcaactttcaactccagttttgaaggtgaaacaggattctcatagagttcttatcactacagaagagggtactattgctgcatcagctgttgtgatggcagtgccattgaatacaatgggagatgttgaatactctcctaggttatcagatgctaagagtgagattgcatcccaaggtcacgctggaaagggagttaaaggatacatcagaattaagcaggatgttggaaatgtgatgacatacgctccagcaaggaacgatgttactccttttacatctgttttcactgatcatgtgggtgaaaatggtactcttctcatagcttttagtgcagatcctaaacttgtggatatcaacgattccaaggctgttgaaaaagcattgcacccacttttgcctggtgttgaagtgacttcttcatatggatacgattggaatcttgatccattttctaagggtacttggtgcacatatagacctggacaaactacaaggtaccttacagaattgcagaaaagagagggtaggcttttctttgcaggaagtgatatggctaacggttggagaggttttattgatggtgctattgaatccggtagggaggttggttatcaggttgcttcatatctcaagggaaagaatagtaacgcataaaatctaagatgttttatcttggttgatcattgtttaatactcctagatagatgggtattcatctatctttttaaaattaattgtcagtacgagtgtttctgagctcaagctt

SEQ ID NO: 7 - protein, 548 aa, Artificial Sequence
  BBLa-PAO fusion construct
MFPLIILISFSLASLSETATGAVTNLSACLINHNVHNFSIYPTSRNYFNLAMANDKGDISKDGVSRRKFLGGAVIGAAAAAGVGSQILSLSATAQGADKERVGPLQSNVDYDAVVIGGGFAGVTAARELSRSGLKTLVLEGRSRLGGRTFTSKLDGEKVELGGTWVHWTQPNVWTEVMHYGLEIEETVGLASPETVIWVTDNQVKRAPAAEAFEIFGAACTEYYKEAHNIYPRPFDPFFAKKALQEMDGLSASEYLNKLSLTREQKDMMDSWLSGNGHNYPETIAYSEIMRWFALSNFNMPTMFDSIARYKIKSGTVSLLEAMVAESDMEVQLSTPVLKVKQDSHRVLITTEEGTIAASAVVMAVPLNTMGDVEYSPRLSDAKSEIASQGHAGKGVKGYIRIKQDVGNVMTYAPARNDVTPFTSVFTDHVGENGTLLIAFSADPKLVDINDSKAVEKALHPLLPGVEVTSSYGYDWNLDPFSKGTWCTYRPGQTTRYLTELQKREGRLFFAGSDMANGWRGFIDGAIESGREVGYQVASYLKGKNSNA

SEQ ID NO: 8 - DNA, 170 bp, Nicotiana tabacum
gaagcagaaatacatacaacatgtttccgctcataattctgatcagcttttcacttgcttccttgtctgaaactgctactggagctgttacaaatctttcagcctgcttaatcaaccacaatgtccataacttctctatttaccccacaagtagaaattactttaacttg

SEQ ID NO: 9 - DNA, 1491 bp, Pseudomonas putida S16
atgacaaaagatggtgatgaaggcagcaaaagcggagtatcacgccgaaagttccttggtagcgccgcggtcggagtggcaacagcgggcatagcctcgcagcttctgactctgtcggcgcccgcggaagcggcggtgaagaccaatgttggtccatcacgcgcaggcgtgggttatgacgttattgtaatcggtggaggcttcgcgggtgttactgcggcgcgagaagcaagccgttctggcttgaaaactctaattcttgaaggtagaagtcggttgggcggccgaacttttacgtctaagcttcagaatcaaaaagttgagttgggaggtacctgggtacattggacccagccgaatgtttggactgagattatgcactatggcctagaggtggaggagacggtcggtcttgcgaatccagagaccgtcatttgggtaacagaggataatgtcaaaagagcacctgcagcagaggcgtttgaaatttttggctctgcctgtaacgaatactacaaagaagcacggaatatttatccgcgcccgtttgaaccattttttgagcgaaagaagctgcagcatgttgatggactttccgcggcagattatcttgaaaaattgcctttgaccagggagcaaaaggatatgatggattcctggctcagtggaaatggacataattatccggaaactattgcatatagcgagattatgcgttggtttgcactcagtaactttaatatgcccacaatgttcgattccattgcgcggtacaaaattaaaaccggtacgcatagtcttttagaggccataatggcggatggtaactcggaagttaaactttcgacgccagtgactaaggttaatcaagataaagataaagtaacagttaccactgaagacggtgtcttcacggcatcagcggtaattgtagccgtccctatcaacactttgcatgatattgagtactcgccgaagttgtcggcagctaaagtggatatgggatcgcaacggcatgctggtgctggtgtaaagggctatattcgcgtaaaacaaaatgtcggcaatgtaatgacatatgctcctgcccggaataagctcacaccatttacctcagtattcacagatcacgtggacgagagcggtacgttactcattgcattttcagccgaccctaagttgattgatatcaatgatatcaaagccgtcgaaaaggctttgcaaccacttttgccaggcgtcgaagtaactgctagttatggctacgactggaacctcgatcccttttctaagggcacttggtgcacttaccgtcctaaccagacgactcgatacttgactgagctgcagaagcgcgaaggacggctattttttgcaggctcggacatggccaatggttggcgtggattcattgatggagcaatcgagaacggtagagaggtagggcatcaagtcgctacgtatctaaaaagagaaaatgacaatgcgtga

SEQ ID NO: 10 - protein, 496 aa, Pseudomonas putida S16
MTKDGDEGSKSGVSRRKFLGSAAVGVATAGIASQLLTLSAPAEAAVKTNVGPSRAGVGYDVIVIGGGFAGVTAAREASRSGLKTLILEGRSRLGGRTFTSKLQNQKVELGGTWVHWTQPNVWTEIMHYGLEVEETVGLANPETVIWVTEDNVKRAPAAEAFEIFGSACNEYYKEARNIYPRPFEPFFERKKLQHVDGLSAADYLEKLPLTREQKDMMDSWLSGNGHNYPETIAYSEIMRWFALSNFNMPTMFDSIARYKIKTGTHSLLEAIMADGNSEVKLSTPVTKVNQDKDKVTVTTEDGVFTASAVIVAVPINTLHDIEYSPKLSAAKVDMGSQRHAGAGVKGYIRVKQNVGNVMTYAPARNKLTPFTSVFTDHVDESGTLLIAFSADPKLIDINDIKAVEKALQPLLPGVEVTASYGYDWNLDPFSKGTWCTYRPNQTTRYLTELQKREGRLFFAGSDMANGWRGFIDGAIENGREVGHQVATYLKRENDNA

(19) United States
(12) Patent Application Publication
(10) Pub. No.: US 2018/0291388 A1
(43) Pub. Date: Oct. 11, 2018

(54) METHODS AND COMPOSITIONS FOR REDUCING THE TOBACCO SPECIFIC NITROSAMINE NNK IN TOBACCO

(71) Applicant: North Carolina State University, Raleigh, NC (US)

(72) Inventors: Ralph E. Dewey, Raleigh, NC (US); Ramsey S. Lewis, Raleigh, NC (US)

(21) Appl. No.: 15/570,963

(22) PCT Filed: May 5, 2016
(86) PCT No.: PCT/US2016/030911

§ 371 Date: Oct. 31, 2017

Related U.S. Application Data
(60) Provisional application No. 62/156,981, filed on May 5, 2015.

(51) Int. Cl. C12N 15/82; A24B 15/10

(52) U.S. Cl. CPC C12N 15/8243; C12Y 104/03024; A24B 15/10

(57) ABSTRACT

The present invention provides a tobacco plant, plant part, and/or plant cell comprising one or more heterologous nucleic acid molecules comprising a nucleotide sequence encoding a pseudooxynicotine degrading enzyme. Further provided are methods and compositions for producing tobacco plants and tobacco products having reduced pseudooxynicotine (PON) and/or 4-(methyl nitrosoamino)-1-(3-pyridyl)-1-butanone (NNK) content.

### STATEMENT OF PRIORITY

This application claims the benefit, under 35 U.S.C. § 119 (e), of U.S. Provisional Application No. 62/156,981; filed May 5, 2015, the entire contents of which is incorporated by reference herein.

### STATEMENT REGARDING ELECTRONIC FILING OF A SEQUENCE LISTING

A Sequence Listing in ASCII text format, submitted under 37 C.F.R. § 1.821, entitled 5051-880WO_ST25.txt, 30,014 bytes in size, generated on May 3, 2016 and filed via EFS-Web, is provided in lieu of a paper copy. This Sequence Listing is hereby incorporated by reference into the specification for its disclosures.

### FIELD OF THE INVENTION

The present invention relates to pseudooxynicotine degrading enzymes and their use in reduction of tobacco specific nitrosamines in tobacco and products therefrom.

### BACKGROUND OF THE INVENTION

Tobacco specific nitrosamines (TSNAs) are a class of compounds produced through the nitrosation of tobacco alkaloids. The levels of TSNAs are very low in freshly harvested tobacco plants, but then increase substantially during the subsequent curing and processing of the leaf (Bush et al. Rec. Adv. Tob. Sci. 27:23-46 (2001)). The four primary TSNAs found in cured tobacco leaves are N′-nitrosonornicotine (NNN), N′-nitrosoanatabine (NAT), N′-nitrosoanabasine (NAB) and 4-(methyl nitrosoamino)-1-(3-pyridyl)-1-butanone (NNK). Interest in TSNAs stems from their involvement in tobacco-associated cancers. Specifically, NNK and NNN are considered among the most potent carcinogens found in tobacco product (Hecht, S. S. Chem. Res. Toxicol. 11:559-603 (1998); Hecht, S. S. Nat. Rev. Cancer 3:733-744 (2003); Hecht, S. S. Langenbecks Arch. Surg. 391: 603-613 (2006); Hoffmann et al., J. Toxicol. Environ. Health 41:1-52 (1994)) and have been classified as Group 1 carcinogens (the strongest classification) by the International Agency for Research on Cancer (IARC, 2007). NAT and NAB, in contrast, appear to be either weakly carcinogenic or benign. Although NNN has been shown to be a potent carcinogen in animal systems, being highly associated with cancers of the esophagus, and oral and nasal cavities, NNK is arguably the most problematic of the TSNAs. A host of carcinogenicity studies in animal systems, combined with epidemiological data and biochemical evaluation of tissues and fluids of smokers, has led researchers to conclude that NNK is a major determinant in tobacco-associated lung cancers (Hecht, S. S. Chem. Res. Toxicol. 11:559-603 (1998); Hecht, S. S. Nat. Rev. Cancer 3:733-744 (2003); Hecht, S. S. Langenbecks Arch. Surg. 391: 603-613 (2006)). In every animal species tested, NNK has been shown to cause lung tumors, regardless of its route of administration. NNK is also likely involved in cancers of the oral and nasal cavities in both smokers and the users of smokeless products alike, as well as cancers of the liver, pancreas and cervix.

TSNAs form when nitrous oxide species (e.g. NO, NO₂, N₂O₃ and N₂O₄) react with tobacco alkaloids (FIG. 1). NAT and NAB are formed via the nitrosation of the secondary alkaloids anatabine and anabasine, respectively. Although early studies claimed that NNN originates from both nicotine and nornicotine (Hecht et al. J. Natl. Cancer Inst. 60:819-824 (1978)), more recent reports have demonstrated that the occurrence of NNN in cured tobacco leaves is strictly correlated with nornicotine content, not nicotine (Bush et al., Rec. Adv. Tob. Sci. 27:23-46 (2001); Lewis et al. Plant Biotech. J. 6:346-354 (2008)). The precursor/product relationship of NNK formation is less clear. A preponderance of the literature simply states that NNK is a nitrosation product of nicotine (e.g., Hoffmann et al., J. Toxicol. Environ. Health 41:1-52 (1994); Hecht, S. S. Chem. Res. Toxicol. 11:559-603 (1998)). This conclusion has been largely based on in vitro observations of the nitrosation properties of nicotine in aqueous environments at low pH (Caldwell et al., Chem. Res. Toxicol. 4:513-516 (1991)). As a tertiary alkaloid possessing a protective methyl group on its pyrrolidine ring, however, nicotine is much less susceptible to nitrosation than the secondary alkaloids nornicotine, anatabine and anabasine that lack similar protective groups on their non-pyridine rings (FIG. 1). Due to the slow reaction rate of nicotine nitrosation, it is likely that an oxidized derivative(s) of nicotine, rather than nicotine itself serves as the direct precursor to NNK (Caldwell et al., Ann. N.Y. Acad. Sci. 686:213-228 (1993)).

Due to the widely accepted presumption that TSNAs significantly contribute toward tobacco-associated cancers, methods to reduce their prevalence in tobacco products have been intensively investigated. In theory, the levels of any TSNA found in tobacco products can be decreased either by the targeted reduction of its alkaloid precursor, or by reducing exposure to the nitrosating agents involved. Among the more notable accomplishment that fall in the latter category include: (1), the modification of flue-curing barns with heat exchangers to reduce the nitrous oxide gases that lead to TSNA formation during leaf cure (Hamm, L. A. Rec. Adv. Tob. Sci. 27:13-15 (2001); Peele et al. Rec. Adv. Tob. Sci. 27:3-12 (2001)); and (2), the use of a pasteurization process to kill nitrite-producing microbes on the leaves of tobacco used for production of the low-TSNA smokeless products referred to as “snus”. The best example of reducing TNSAs through the targeted reduction of an alkaloid precursor involves recent efforts aimed at lowering the NNN content of cured tobacco leaves. Through knocking out the function of genes responsible for the synthesis of alkaloid nornicotine, it was demonstrated that nornicotine and NNN levels in air-cured burley tobaccos could be reduced by about 80% in comparison to that achievable using existing commercial varieties and production practices (Lewis et al. Plant Biotech. J. 6:346-354 (2008); Lewis et al. Phytochemistry 71:1988-1998 (2010)). Although significant progress has been made over the past two decades in lowering the amounts of TNSAs that form and accumulate within the cured tobacco leaf, the fact remains that the amounts of NNK and NNN found in most commercial tobacco products, are still hundreds to thousands of times higher than the levels of carcinogenic nitrosamines found in other consumables, such as cured meat, beer and other foods (Bartsch and Spiegelhalder, Eur. J. Can. Prevent. 5, 11-18 (1996); Hecht et al., Tob. Control 20, 443 (2011); Hotchkiss, J. H. Cancer Surv. 8:295-321(1989)). Therefore, there still exists a great need to devise methodologies that can further reduce the levels of TNSAs, particularly NNK, in tobacco plants and products produced from tobacco plants.

### SUMMARY OF THE INVENTION

In one aspect, the present invention provides a tobacco plant, plant part, and/or plant cell comprising one or more heterologous nucleic acid molecules comprising a nucleotide sequence encoding a pseudooxynicotine (PON) degrading enzyme. In some aspects, the nucleotide sequence encoding the PON degrading enzyme can be optimized for expression in tobacco. In additional aspects, the PON degrading enzyme can be fused (i.e., operably linked) to a vacuolar targeting sequence or to an endoplasmic reticulum targeting signal sequence. In particular aspects, the PON degrading enzyme is a pseudooxynicotine amine oxidase (PAO).

In a further aspect, the present invention provides a method of reducing PON and/or NNK in a tobacco plant comprising: introducing into a tobacco plant, plant part and/or plant cell one or more heterologous nucleic acid molecules comprising a nucleotide sequence encoding a PON degrading enzyme.

In a still further aspect, a method of producing a plant, plant part, or plant cell having reduced PON and/or NNK content is provided, the method comprising: introducing into a tobacco plant, plant part and/or plant cell one or more heterologous nucleic acid molecules comprising a nucleotide sequence encoding a PON degrading enzyme, thereby producing a tobacco plant, plant part and/or plant cell having reduced PON and/or NNK content.

In an additional aspect, the present invention provides a method of producing a tobacco product having reduced NNK content, the method comprising: introducing into a tobacco plant, plant part and/or plant cell one or more heterologous nucleic acid molecules comprising a nucleotide sequence encoding a PON degrading enzyme, thereby producing a transgenic tobacco plant, plant part and/or plant cell having reduced PON and/or NNK content and producing a tobacco product from said transgenic tobacco plant, plant part and/or plant cell, wherein the tobacco product has reduced PON and/or NNK content.

In a further aspect, the present invention provides a method of producing a tobacco product having reduced NNK content, the method comprising: producing a tobacco product from a tobacco plant, plant part and/or plant cell of the invention, said tobacco plant, plant part and/or plant cell comprising one or more heterologous nucleic acid molecules comprising a nucleotide sequence encoding a PON degrading enzyme, wherein the tobacco product has reduced PON and/or NNK content.

Additional aspects of the invention provide compositions including nucleic acid constructs comprising a nucleotide sequence encoding a pseudooxynicotine (PON) degrading enzyme for transforming a tobacco plant, plant part and/or plant cell. Also provided herein are transformed tobacco plant cells, plants and/or plant parts as well as progeny plants, harvested and processed products produced from said transformed plant cell, plant, plant parts, and/or progeny plants.

These and other aspects of the invention are set forth in more detail in the description of the invention below.

### BRIEF DESCRIPTION OF THE DRAWINGS

FIG. 1 shows the precursor/product relationships between tobacco alkaloids and TSNAs.

FIG. 2A-2G shows nucleotide and predicted amino acid sequences relevant to this study. FIG. 2A shows the nucleotide sequence of the pseudooxynicotine amine oxidase (PAO) gene as found in Pseudomonas strain HZN6 (GenBank accession #JN391188) (SEQ ID NO:1). Start and stop codons are underlined. FIG. 2B shows the nucleotide sequence of the PAO gene (SEQ ID NO:2) used to transform tobacco cultivars K326 SRC and TN90 SRC. The Pseudomonas HZN6 PAO sequence optimized for expression in tobacco is shown in black; 5′ and 3′ UTR sequences obtained from the tobacco CYP82E10 gene are shown in bold; nucleotides engineered to create restriction sites to facilitate cloning are shown in lowercase. Start and stop codons are underlined. FIG. 2C shows the predicted amino acid sequence of PAO (SEQ ID NO:3). FIG. 2D shows the nucleotide sequence of the tobacco BBLa gene (GenBank Accession #AB604219) (SEQ ID NO:4). Start and stop codons are underlined. FIG. 2E. shows the predicted amino acid sequence of BBLa (SEQ ID NO:5).

FIG. 2F shows the nucleotide sequence of the BBL_{vac}-PAO fusion construct (SEQ ID NO:6). Black, italicized sequences correspond to the BBLa 5′ flanking sequence and first 50 codons that contain a vacuolar localization signal; black, standard font sequences represent the optimized Pseudomonas HZN6 PAO sequence; 3′ UTR sequence obtained from the tobacco CYP82E10 gene is shown in bold; nucleotides engineered to create restriction sites to facilitate cloning are shown in lowercase. Start and stop codons are underlined. FIG. 2G shows the predicted amino acid sequence of BBL_{vac}-PAO fusion protein (SEQ ID NO:7). Amino acids obtained from BBLa are shown in italicized red; residues shown in black correspond to PAO; the bold, underlined alanine residue was generated as a consequence of creating the fusion construct.

FIG. 3 shows the nicotine, NNK, NNN and NAT concentrations in cured leaves of control plants (E4:GUS) and plants containing a PAO construct. Values shown represent the means±standard errors of 6-10 T₀ plants for each genotype. Genotypes containing PAO constructs whose means are not significantly different (P<0.05) from the E4:GUS control are indicted with an “a”; means that are significantly different from the control genotype are shown with a “b”.

FIG. 4 shows the fraction of total TSNAs present as NNK in the air-cured leaf of TN90 SRC and three T₂ transgenic lines expressing the PAO transgene within the same background. Bars represent the means of 30 plants for each genotype. P values for T-tests for differences between the transgenic line mean and the mean of the non-transgenic TN90 SRC control are: 35S:PAO#11, P=0.0084; 35S:PAO#3, P=0.0550; 35S:BBLvac-PAO#3, P=0.0537.

### DETAILED DESCRIPTION OF THE INVENTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein, when referring to a measurable value such as an amount or a time period and the like, encompasses variations of 20%, +10%, +5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The terms “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

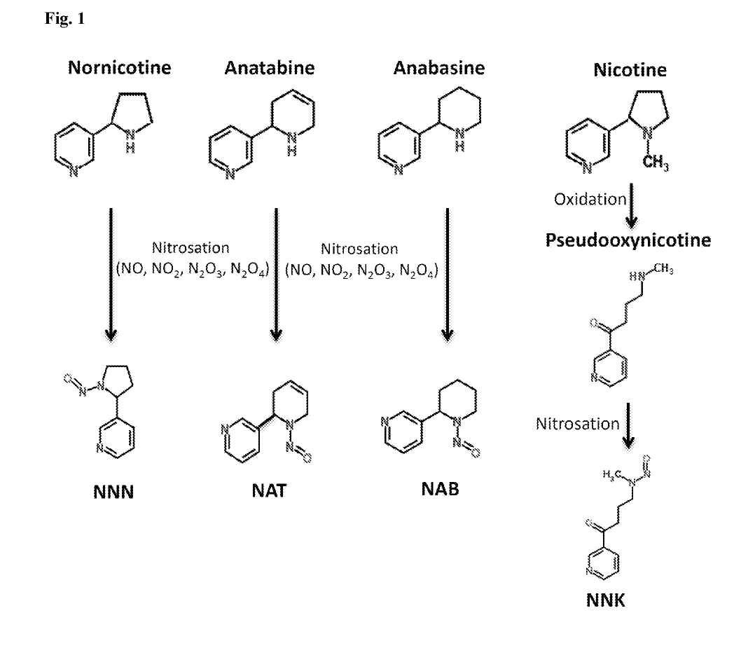

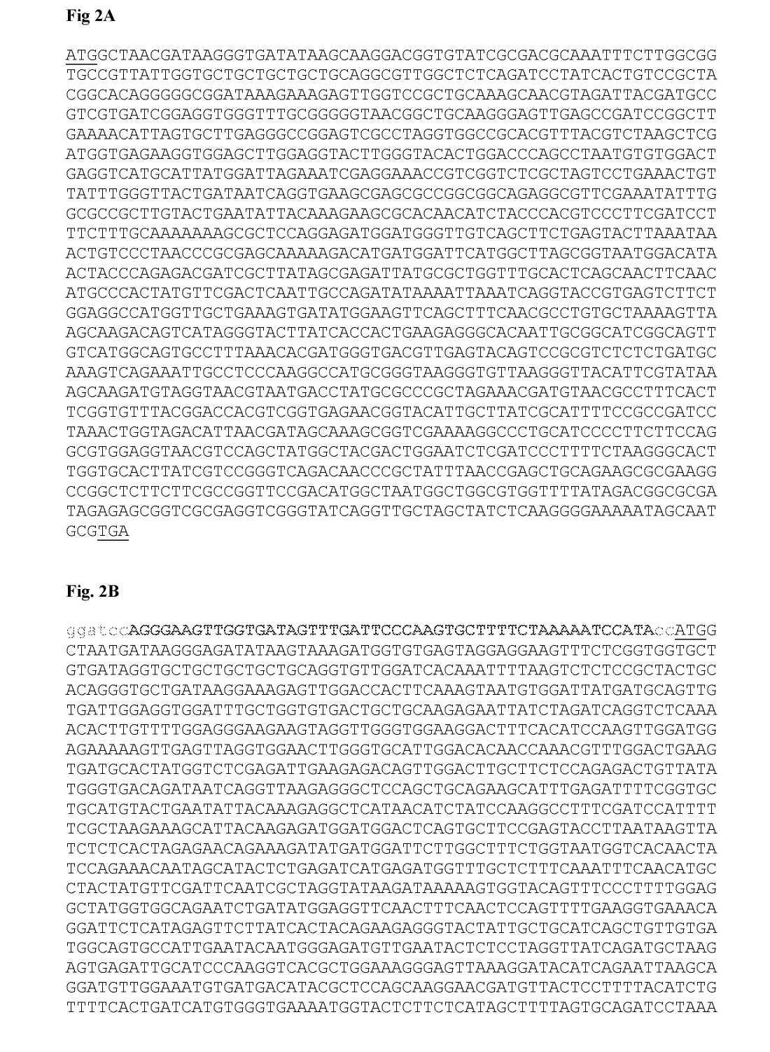

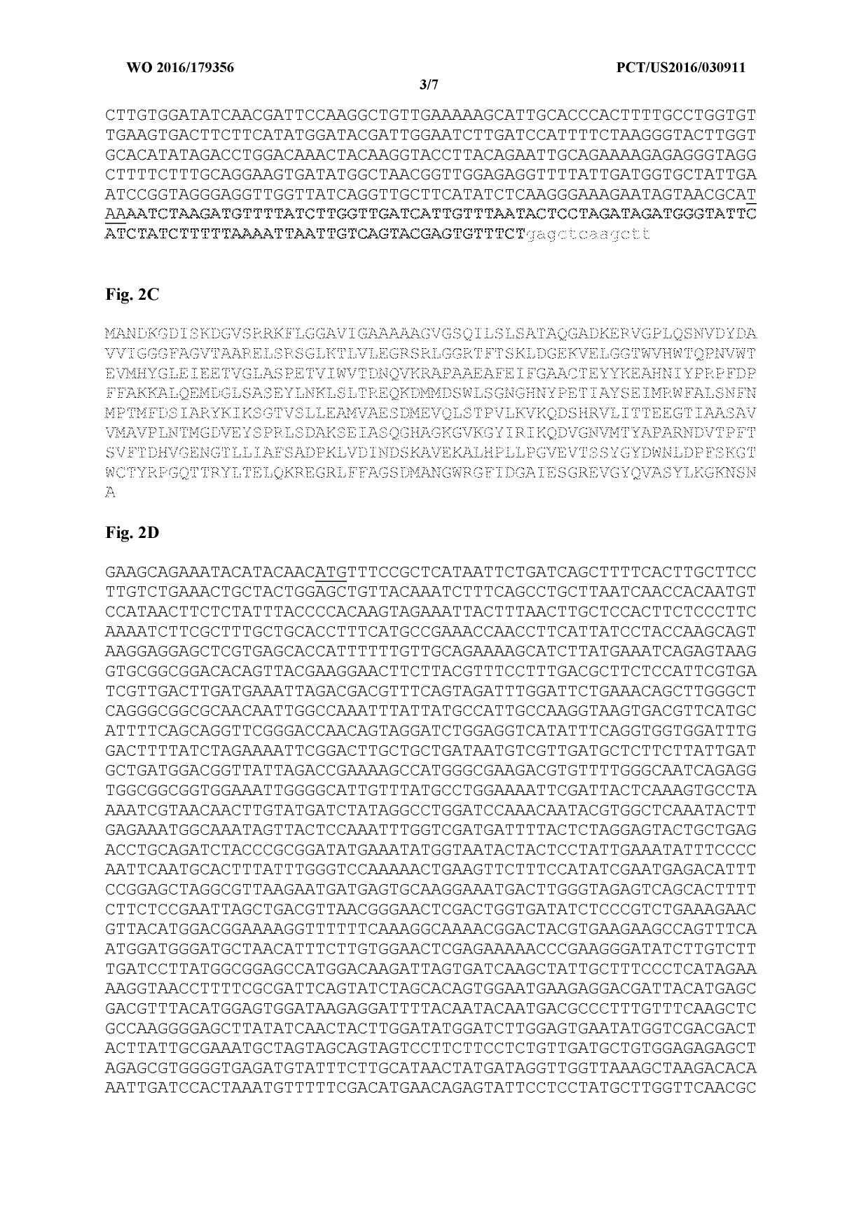

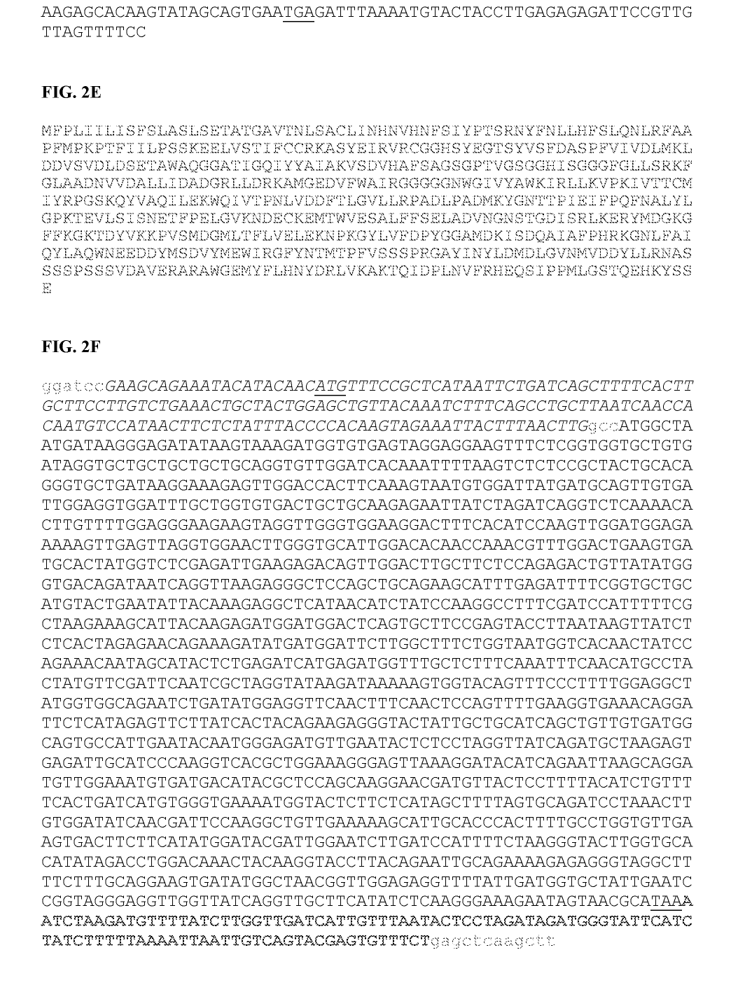

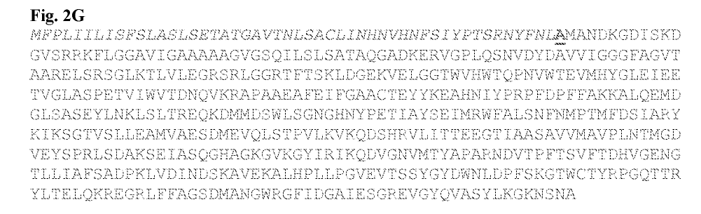

The present invention is directed to the reduction of TSNAs in tobacco, more particularly, a reduction in NNK. Given the chemical stability of nicotine, it is highly likely that one or more oxidative derivative of nicotine is the direct alkaloid precursor of NNK, as opposed to nicotine per se. Of the several known oxidized nicotine derivatives, the compound PON is the one most likely to serve as a precursor to NNK, having a structure identical to NNK, but with the absence of a nitric oxide group (the unit added upon nitrosation) on its secondary nitrogen (FIG. 1). Although few would dispute that PON could physically serve as a precursor to NNK, early investigations questioned its relevance in NNK formation in vivo as PON could not be definitively shown to be present in measurable amounts in tobacco extracts (for example, see tobacco legacy documents at tobaccodocuments.org/ahf/2023321204-1214.html). A key advance in this field came with the discovery that PON exists in equilibrium with three other molecular species: 2′-hydroxynicotine, N′-methylmyosmine and nicotine 1′, 2′-iminium ion (Wei, Studies on the biosynthesis and metabolites of pyridine alkaloids in Nicotiana species. Ph.D. Thesis. Univ. of Kentucky (2000)). The ability of PON to rapidly interchange molecular species, coupled with its high sensitivity to oxidation during extraction, made it particularly difficult to accurately purify and quantitate from tobacco tissue. However, it was determined that nicotine 1′, 2′-iminium ions could be trapped using potassium cyanide to produce 2′-cyanonicotine (Neurath et al. Med. Sci. Res. 20:853-858 (1992)), and from this a protocol was developed whereby PON levels could be accurately quantified from tobacco leaf materials through the indirect quantification of 2′-cyanonicotine using Gas Chromatography/Mass Spectroscopy (Wei, Studies on the biosynthesis and metabolites of pyridine alkaloids in Nicotiana species. Ph.D. Thesis. Univ. of Kentucky (2000)). Using this methodology, it was not only confirmed that PON was indeed present in both green and cured tobaccos, but that it existed in sufficient quantities to serve as the alkaloid precursor for NNK.

Accordingly, in one aspect, the present invention provides a tobacco plant, plant part, and/or plant cell comprising one or more heterologous nucleic acid molecules comprising, consisting essentially of, or consisting of a nucleotide sequence encoding a pseudooxynicotine (PON) degrading enzyme.

Another aspect of the present invention provides a method of reducing PON and/or NNK content in a tobacco plant and/or plant part comprising: introducing into a tobacco plant, plant part and/or plant cell one or more heterologous nucleic acid molecule comprising, consisting essentially of, or consisting of a nucleotide sequence encoding a PON degrading enzyme, thereby reducing the PON and/or NNK content in the transgenic tobacco plant and/or plant part as compared to a tobacco plant, plant part and/or plant cell not transformed with said one or more heterologous nucleic acid molecules. In some embodiments, the PON content can be reduced by about 10% to about 100% as compared to a control (e.g., wild type, a tobacco plant not comprising a nucleotide sequence encoding a PON degrading enzyme). Thus, for example, the PON content in a tobacco plant and/or plant part of this invention can be reduced by about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any range or value therein as compared to a control. In representative embodiments, the PON content can be reduced by about 10% to about 50%, about 20% to about 50%, about 20% to about 60%, about 20% to about 80%, about 20% to about 90%, about 30% to about 60%, about 30% to about 80% about 30% to about 95%, and the like, as compared to a control. In additional embodiments of the invention, the NNK content in a tobacco plant and/or plant part of this invention can be reduced by about 10% to about 100% as compared to a control (e.g., wild type, a tobacco plant not comprising a nucleotide sequence encoding a PON degrading enzyme). Thus, in some embodiments, the NNK content can be reduced by about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any range or value therein as compared to a control. In representative embodiments, the NNK content can be reduced by about 10% to about 50%, about 20% to about 50%, about 20% to about 60%, about 20% to about 80%, about 20% to about 90%, about 30% to about 60%, about 30% to about 80% about 30% to about 95%, and the like as, compared to a control.

A further aspect of the invention provides a method of producing a plant, plant part, or plant cell having reduced PON and/or NNK content, comprising: introducing into a tobacco plant, plant part and/or plant cell one or more heterologous nucleic acid molecules comprising, consisting essentially of, or consisting of a nucleotide sequence encoding a PON degrading enzyme, thereby producing a tobacco plant, plant part and/or plant cell having reduced PON and/or NNK content as compared to a tobacco plant, plant part and/or plant cell not transformed with said one or more heterologous nucleic acid molecules.

In an additional aspect, the present invention provides a method of producing a tobacco product having reduced PON and/or NNK content, the method comprising: producing a tobacco product from a tobacco plant, plant part and/or plant cell of the invention, said tobacco plant, plant part and/or plant cell comprising one or more heterologous nucleic acid molecules comprising a nucleotide sequence encoding a PON degrading enzyme, wherein the tobacco product has reduced PON and/or NNK content.

In still further aspects, the present invention provides a method of producing a tobacco product having reduced PON and/or NNK content, the method comprising: introducing into a tobacco plant, plant part and/or plant cell one or more heterologous nucleic acid molecules comprising, consisting essentially of, or consisting of a nucleotide sequence encoding a PON degrading enzyme, thereby producing a transgenic tobacco plant, plant part and/or plant cell having reduced PON and/or NNK content as compared to a tobacco plant, plant part and/or plant cell not transformed with said one or more heterologous nucleic acid molecules; and producing a tobacco product from said transgenic tobacco plant, plant part and/or plant cell, wherein the tobacco product has reduced PON and/or NNK content as compared to a tobacco product produced from a plant, plant part and/or plant cell not transformed with said one or more heterologous nucleic acid molecules.

In some embodiments, the PON content of a product produced from a tobacco plant and/or plant part of this invention can be reduced by about 10% to about 100% as compared to a control (e.g., wild type, a tobacco plant not comprising a nucleotide sequence encoding a PON degrading enzyme). Thus, for example, the PON content can be reduced by about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any range or value therein as compared to a control. In representative embodiments, the PON content can be reduced by about 10% to about 50%, about 20% to about 50%, about 20% to about 60%, about 20% to about 80%, about 20% to about 90%, about 30% to about 60%, about 30% to about 80% about 30% to about 95%, and the like, as compared to a control. In additional embodiments of the invention, the NNK content of a product produced from a tobacco plant and/or plant part of this invention can be reduced by about 10% to about 100% as compared to a control (e.g., wild type, a tobacco plant not comprising a nucleotide sequence encoding a PON degrading enzyme). Thus, in some embodiments, the NNK content can be reduced by about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any range or value therein as compared to a control. In representative embodiments, the NNK content can be reduced by about 10% to about 50%, about 20% to about 50%, about 20% to about 60%, about 20% to about 80%, about 20% to about 90%, about 30% to about 60%, about 30% to about 80% about 30% to about 95%, and the like as, compared to a control.

In some aspects, the PON degrading enzyme can be a microbial enzyme obtained from, for example, a bacteria or fungus known to degrade nicotine. In particular aspects, the fungal genera can be Aspergillis. In other aspects, the bacterial genera can be Arthrobacter, Agrobacterium or Pseudomonas. As would be well understood in the art, a plant can be transformed with and express more than one heterologous nucleic acid encoding a PON degrading enzyme, wherein the PON degrading enzymes can be from same or any combination of organisms (e.g., bacterial, fungal, Aspergillis, Arthrobacter, Agrobacterium, Pseudomonas and the like).

In some aspects of the invention, a PON degrading enzyme can be a pseudooxynicotine amine oxidase (PAO). In representative embodiments, a PAO can be from a bacterium. In some embodiments, the bacterium can be Pseudomonas.

Thus, for example, in 2012, Qiu et al. reported on the characterization of two novel genes from a Pseudomonas strain (HZN6) that is capable of metabolizing nicotine (Appl. Environ. Microbiol. 78:2154-2160). One of these genes was shown to encode pseudooxynicotine amine oxidase (PAO), that converts PON to 3-succinoylsemialdehyde-pyridine and methylamine. The present inventors surprisingly discovered that expression of PAO in tobacco plants led to the metabolism of PON with a resultant decrease in the levels of this important NNK precursor. In some embodiments, the nucleotide sequence encoding PAO from Pseudomonas strain HZN6 comprises, consists essentially of, or consists of the nucleotide sequence of SEQ ID NO:1. In still other embodiments, the nucleotide sequence encoding PAO from Pseudomonas strain HZN6 encodes a polypeptide comprising, consisting essentially of, or consisting of the amino acid sequence of SEQ ID NO:3. In a further example, the PON degrading enzyme can be from another nicotine degrading strain, Pseudomonas putida S16. The entire genome of P. putida S16 has been sequenced (Hongzhi, et al., 2011. J. Bacteriol. 193:5541-5542) and a candidate PAO has been identified having at least 79% identity to the PAO from Pseudomonas strain HZN6 (e.g., nucleotide sequence of SEQ ID NO:9; amino acid sequence of SEQ ID NO:10). Accordingly, in some embodiments, one or more heterologous nucleic acids encoding a PON degrading enzyme can comprise, consist essentially of, or consist of the nucleotide sequence of SEQ ID NO:1 or 9 or can comprise, consist essentially of, or consist of a nucleotide sequence encoding the amino acid sequence of SEQ ID NO:3 or 10.

In some aspects, a nucleotide sequence encoding a PON degrading enzyme can be codon optimized for expression in tobacco.

Non-limiting examples of such nucleotide sequence modifications include an altered G/C content to more closely approach that typically found in the species of interest (i.e., tobacco), and the removal of codons atypically found in, for example, tobacco.

Thus, the phrase “codon optimization,” as used herein, refers to the selection of appropriate DNA nucleotides for use within a structural gene or fragment thereof that approaches codon usage within tobacco. Therefore, an optimized gene or nucleic acid sequence refers to a nucleotide sequence of a native or naturally occurring gene that has been modified to utilize statistically-preferred or statistically-favored codons within the species of interest. The nucleotide sequence typically is examined at the DNA level and the coding region optimized for expression in said species determined using any suitable procedure, for example as described in Sardana et al. (1996, Plant Cell Reports 15:677-681) (see also, U.S. Pat. No. 8,513,488 and WO/1993/007278). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1 SDCU=n=1N[(Xn−Yn)/Yn]2/N, where Xn refers to the frequency of usage of codon n in highly expressed plant genes, where Yn to the frequency of usage of codon n in the gene of interest and N refers to the total number of codons in the gene of interest (U.S. Pat. No. 8,513,488). Codon usage from highly expressed genes of dicotyledonous plants can be found in Murray et al. (1989, Nuc Acids Res. 17:477-498).

Another method of optimizing a nucleic acid sequence in accordance with the preferred codon usage for a particular plant cell type is based on the direct use, without performing any extra statistical calculations, of codon optimization tables such as those provided on-line at the Codon Usage Database through the NIAS (National Institute of Agrobiological Sciences) DNA bank in Japan (http://www.kazusa.or.jp/codon/).

Thus, in particular embodiments, the PON degrading enzyme can be a PAO optimized for expression in tobacco, wherein the optimized nucleotide sequence encoding PAO comprises the nucleotide sequence of SEQ ID NO:2.

The present inventors have further discovered that the efficiency of PON degradation in tobacco cells can be increased by targeting the PON degrading enzyme to the vacuole. Thus, in some aspects of the invention, the nucleotide sequence encoding the PON degrading enzyme (e.g., a PON degrading enzyme from a fungal or bacterial genera known to degrade nicotine; a pseudooxynicotine amine oxidase (PAO); and/or a PAO from Pseudomonas strain HZN6 or Pseudomonas putida S16) can be fused to a vacuolar targeting sequence. Vacuolar targeting or sorting sequences (VSS) are well known in the art and can comprise three distinct types, ones located at the N-terminus, the C-terminus, or those internal to the protein (see, e.g., Neuhaus and Rogers, Plant Mol Biol. 38:127-144 (1998); Misaki et al. J. Biosci Bioengin 112 (5):476-484 (2011); Vitale and Raikhel Trends in Plant Sci 4(4):149-155 (1999)). Thus, any vacuolar targeting sequence known or later discovered can be used to target the PON degrading enzyme of this invention to the vacuole of the cell of the tobacco plant. In some embodiments of this invention, the vacuolar targeting sequence can be from tobacco. In other embodiments, the vacuolar targeting sequence is from a plant other than tobacco.

Some exemplary vacuolar targeting sequences include the vacuolar targeting sequence from: a berberine bridge-like enzyme; sweet potato prosporamine; barley proaleurain; barley lectin; tobacco chitinase; tobacco glucanase; tobacco osmotin; Brazil nut and pea 2S albumin storage proteins; castor bean ricin; and/or common bean phaseolin.

In representative embodiments, a PON degrading enzyme can be targeted to the vacuole using a vacuolar targeting or sorting sequence from a tobacco berberine bridge-like enzyme. Tobacco berberine bridge-like enzymes, include, but are not limited to, BBLa, BBLb, BBLc and BBLd (Kajikawa et al. Plant Physiol. 155:2010-2022 (2011)). In some embodiments, the nucleotide sequence encoding the berberine bridge-like enzyme of BBLa can be the nucleotide sequence of SEQ ID NO:4. In other embodiments, the nucleotide sequence encoding the berberine bridge-like enzyme of BBLa can be a nucleotide sequence encoding a polypeptide having the amino acid sequence of SEQ ID NO:5. In still further embodiments, the vacuolar targeting sequence from a berberine bridge-like enzyme comprises the nucleotide sequence of SEQ ID NO:8.

In some embodiments, a nucleotide sequence encoding a PON degrading enzyme fused to a vacuolar targeting sequence comprises, consists essentially of, or consists of the nucleotide sequence of SEQ ID NO:6, wherein the PON degrading enzyme is a PAO from Pseudomonas strain HZN6. In further embodiments, a nucleotide sequence encoding a PON degrading enzyme fused to a vacuolar targeting sequence encodes a polypeptide having the amino acid sequence of SEQ ID NO:7, wherein the PON degrading enzyme is a PAO from Pseudomonas strain HZN6 and the vacuolar targeting sequence is from a tobacco berberine bridge-like enzyme (e.g., BBLa).

Additionally, to reduce the level of extracellular pools of PON and NNK, it may be advantageous to direct a nucleotide sequence encoding the PON degrading enzyme to be transported to the apoplast. Thus, in some embodiments, a nucleotide sequence encoding the PON degrading enzyme (e.g., a PON degrading enzyme from a fungal or bacterial genus/species known to degrade nicotine; a pseudooxynicotine amine oxidase (PAO); and/or a PAO from Pseudomonas strain HZN6 or Pseudomonas putida S16) can be targeted to the apoplast). Targeting proteins to the apoplast can be initiated through the placement of a cleavable signal sequence at the N-terminus of the protein (e.g., at the N-terminus of the PON degrading enzyme) to direct its passage through the endoplasmic reticulum (ER), thus introducing the protein to the secretory pathway. In the absence of additional signals to facilitate either localization to the vacuole or retention within the ER or the Golgi, proteins directed through the secretory pathway are typically transported to the apoplast by a “default pathway” (Hood et al., In A. Altman and P. M. Hasegawa, eds. Plant Biotechnology and Agriculture. Chapter 3. Academic Press, pp. 35-54 (2012)). Thus, in some embodiments, ER retention signals are not present.

Thus, in some embodiments, the PON degrading enzyme that is to be directed to the apoplast can be fused to an ER targeting signal sequence. ER targeting signal sequences are well known in the art (see, e.g., Hood et al., In A. Altman and P. M. Hasegawa, eds. Plant Biotechnology and Agriculture. Chapter 3. Academic Press, pp. 35-54 (2012)) and any ER targeting signal sequence known or later discovered can be used to target the PON degrading enzyme of this invention to the apoplast of the cell of a tobacco plant. In some embodiments of this invention, the ER targeting signal sequence can be from tobacco. In other embodiments, the ER targeting signal sequence can be from a plant other than tobacco.

Exemplary ER targeting signal sequences include, but are not limited to, ER targeting signal sequences from extensin, osmotin, osmotin-like proteins, PR-S, PR1a, barley alpha amylase, cecropin, chitinase A, E1 endoglucanase, and/or sporamin.

In some embodiments, a tobacco plant, plant part, and/or plant cell is provided that comprises at least two least two heterologous nucleic acid molecules comprising a nucleotide sequence encoding a PON degrading enzyme, wherein at least one of the at least two heterologous nucleic acid molecules comprises a nucleotide sequence encoding a PON degrading enzyme fused to a vacuolar targeting sequence and at least one of the at least two heterologous nucleic acid molecules comprises a nucleotide sequence encoding a PON degrading enzyme fused to a ER targeting signal sequence.

A nucleic acid molecule or expression cassette of this invention comprising a polynucleotide encoding a PON degrading enzyme can further comprise one or more regulatory sequences. Accordingly, in some embodiments, the present invention provides nucleic acid construct comprising in the 5′ to 3′ direction, a promoter operable in a plant cell and the nucleic acid molecule of the invention positioned downstream from said promoter and operatively associated therewith. In other embodiments, a regulatory sequence can be a promoter, a 5′ untranslated region (UTR), a 3′ UTR, a termination sequence, or any combination thereof.

Additional embodiments of this invention provide a progeny plant produced from the transgenic plant, plant part or plant cell of this invention, wherein said progeny plant comprises the one or more heterologous nucleic acid molecules comprising, consisting essentially of, or consisting of a nucleotide sequence encoding a pseudooxynicotine (PON) degrading enzyme. Still other embodiments provide a seed from a transgenic plant or a progeny plant of this invention, wherein said seed comprises in its genome one or more heterologous nucleic acid molecules comprising a nucleotide sequence encoding a pseudooxynicotine (PON) degrading enzyme. Further embodiments provide a plant produced from a seed of the invention.

The invention further provides a plant crop comprising a plurality of transgenic tobacco plants of the invention planted together in an agricultural field.

In additional embodiments, the present invention provides a tobacco product produced from the transgenic tobacco plants, plant parts, plant cells, the progeny plants thereof and/or crops thereof, wherein the product has a reduced amount of PON and/or NNK. A tobacco product can be any product made using the tobacco plants, plant parts or plant cells of this invention. Accordingly a tobacco product can be leaf tobacco, shredded tobacco, cut tobacco, ground tobacco, powder tobacco, tobacco extract, a nicotine extract (e.g., nicotine extracted from tobacco of this invention for use in, for example, e-cigarettes or nicotine replacement therapy products (e.g., gums, lozenges, patches, and the like)), smokeless tobacco, moist or dry snuff, kretek, pipe tobacco, cigar tobacco, cigarillo tobacco, cigarette tobacco, chewing tobacco, bidis, bits, and tobacco-containing gum, lozenges, or any combination thereof. In other embodiments, the tobacco product can be a cigarillo, a non-ventilated recess filter cigarette, a vented recess filter cigarette, a cigar, snuff, chewing tobacco, or any combination thereof. In some embodiments, the tobacco extract made from the transgenic tobacco plants, plant parts or plant cells of this invention can be used in electronic cigarettes.

As described herein, the present invention is directed to methods and compositions for the reduction of PON in tobacco plants, parts and/or cells and products produced therefrom, thereby reducing the amount of PON and/or NNK in said tobacco plants, parts and/or cells and tobacco products obtained therefrom. The term “tobacco” as used herein means any plant of the genus Nicotiana. Thus, the term “tobacco” can include, but is not limited to, Nicotiana L., N. tabacum, N. benthamiana, N. rustica, N. alata, N. sylvestris, N. acuminata, N. bigelovii, N. obtusifolia, N. quadrivalvis, N. trigonophylla, N. affinis, N attenuata, N. clevelandii, N. excelsior, N. forgetiana, N. glauca, N. glutinosa, N. langsdorffii, N. longiflora, N. obtusifolia, N. trigonophylla, N. palmeri, N. paniculata, N. plumbaginifolia, N. quadrivalvis, N. repanda, N. suaveolens, N. sylvestris, N. tomentosa, N. velutina and the like. Further, any variety of tobacco for which reduction in TSNAs, in particular, a reduction in NNK, is desirable is useful with this invention.

Tobacco “plant part,” as used herein, includes but is not limited to reproductive tissues (e.g., petals, sepals, stamens, pistils, receptacles, anthers, pollen, flowers, fruits, flower bud, ovules, seeds, embryos, capsules); vegetative tissues (e.g., petioles, stems, roots, root hairs, root tips, pith, wood, coleoptiles, stalks, shoots, branches, bark, apical meristem, axillary bud, cotyledon, hypocotyls, roots, root tips, trichomes, leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the tobacco plant, which typically comprise a cell wall but also includes protoplasts. A tobacco plant cell of the present invention can be in the form of an isolated single cell or can be a cultured cell or can be a part of a higher-organized unit such as, for example, a plant tissue (including callus) or a plant organ. A “protoplast” is an isolated plant cell without a cell wall or with only parts of the cell wall. Thus, in some embodiments of the invention, a transgenic cell comprising a nucleic acid molecule and/or nucleotide sequence of the invention is a cell of any plant or plant part including, but not limited to, a root cell, a leaf cell, a tissue culture cell, a seed cell, a flower cell, a fruit cell, a pollen cell, and the like.

“Plant cell culture” means cultures of plant units such as, for example, protoplasts, cell culture cells, cells in plant tissues, pollen, pollen tubes, ovules, embryo sacs, zygotes and embryos at various stages of development. In some embodiments of the invention, a transgenic tissue culture or transgenic plant cell culture is provided, wherein the transgenic tissue or cell culture comprises a nucleic acid molecule/nucleotide sequence of the invention.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or a functional untranslated RNA. A “functional” RNA includes any untranslated RNA that has a biological function in a cell, e.g., regulation of gene expression. Such functional RNAs include but are not limited to RNAi (e.g., siRNA, shRNA, antisense RNA), miRNA, ribozymes, RNA aptamers, and the like.

As used herein, the terms “reduce,” “reduced,” “reducing,” “reduction,” “diminish,” “suppress,” and “decrease” (and grammatical variations thereof), describe, for example, a decrease in PON and/or NNK content in a tobacco plant, plant part, or plant cell comprising in its genome a heterologous nucleic acid molecule comprising a nucleotide sequence encoding a PON degrading enzyme as compared to a control plant, plant part, or plant cell that does not comprise in its genome said heterologous nucleic acid molecule comprising a nucleotide sequence encoding a PON degrading enzyme. Thus, as used herein, the terms “reduce,” “reduces,” “reduced,” “reduction,” “diminish,” “suppress,” and “decrease” and similar terms mean a decrease of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, and the like, or more, or any range therein, as compared to a control (e.g., a plant, plant part, or plant cell that does not comprise in its genome said heterologous nucleic acid molecule comprising a nucleotide sequence encoding a PON degrading enzyme).

An “isolated” nucleic acid molecule, an “isolated” nucleotide sequence or an “isolated” polypeptide is a nucleic acid molecule, nucleotide sequence or polypeptide that, by the hand of man, exists apart from its native environment and is therefore not a product of nature. An isolated nucleic acid molecule, nucleotide sequence or polypeptide may exist in a purified form that is at least partially separated from at least some of the other components of the naturally occurring organism or virus, for example, the cell or viral structural components or other polypeptides or nucleic acids commonly found associated with the polynucleotide. In representative embodiments, the isolated nucleic acid molecule, the isolated nucleotide sequence and/or the isolated polypeptide is at least about 1%, 5%, 10%, 20%, 30%, 40%, 50%, 60%, 70%, 80%, 90%, 95%, or more pure.

In other embodiments, an isolated nucleic acid molecule, nucleotide sequence or polypeptide may exist in a non-native environment such as, for example, a recombinant host cell. Thus, for example, with respect to nucleotide sequences, the term “isolated” means that it is separated from the chromosome and/or cell in which it naturally occurs. A polynucleotide is also isolated if it is separated from the chromosome and/or cell in which it naturally occurs in and is then inserted into a genetic context, a chromosome and/or a cell in which it does not naturally occur (e.g., a different host cell, different regulatory sequences, and/or different position in the genome than as found in nature). Accordingly, the recombinant nucleic acid molecules, nucleotide sequences and their encoded polypeptides are “isolated” in that, by the hand of man, they exist apart from their native environment and therefore are not products of nature, however, in some embodiments, they can be introduced into and exist in a recombinant host cell.

A “heterologous” nucleotide sequence, nucleic acid or polypeptide is a nucleotide sequence not naturally associated with a host cell into which it is introduced, including non-naturally occurring multiple copies of a naturally occurring nucleotide sequence or a naturally occurring nucleotide sequence that is introduced into a non-natural genomic context (e.g., different chromosome, a different location on the same chromosome and/or with different regulatory elements).

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the organism. A “homologous” nucleic acid sequence is a nucleotide sequence naturally associated with a host cell into which it is introduced.

Also as used herein, the terms “nucleic acid,” “nucleic acid molecule,” “nucleotide sequence” and “polynucleotide” can be used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of this invention has a significant sequence identity (e.g., 70%, 75%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to the nucleotide sequences of the invention.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

As used herein, the phrase “substantially identical,” in the context of two nucleic acid molecules, nucleotide sequences or protein sequences, refers to two or more sequences or subsequences that have at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 95%, at least about 96%, at least about 97%, at least about 98%, or at least about 99% nucleotide or amino acid residue identity, when compared and aligned for maximum correspondence, as measured using one of the following sequence comparison algorithms or by visual inspection. In some embodiments of the invention, the substantial identity exists over a region of the sequences that is at least about 50 residues to about 150 residues in length. Thus, in some embodiments of the invention, the substantial identity exists over a region of the sequences that is at least about 50, about 60, about 70, about 80, about 90, about 100, about 110, about 120, about 130, about 140, about 150, or more residues in length. In some particular embodiments, the sequences are substantially identical over at least about 150 residues. In a further embodiment, the sequences are substantially identical over the entire length of the coding regions. Furthermore, in representative embodiments, substantially identical nucleotide or protein sequences perform substantially the same function (e.g., degrading PON or reducing PON content).

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

Two nucleotide sequences can be considered to be substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0M Na ion, typically about 0.01 to 1.0M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

The following are examples of sets of hybridization/wash conditions that may be used to clone homologous nucleotide sequences that are substantially identical to reference nucleotide sequences of the invention. In one embodiment, a reference nucleotide sequence hybridizes to the “test” nucleotide sequence in 7% sodium dodecyl sulfate (SDS), 0.5M NaPO₄, 1 mM EDTA at 50° C. with washing in 2×SSC, 0.1% SDS at 50° C. In another embodiment, the reference nucleotide sequence hybridizes to the “test” nucleotide sequence in 7% sodium dodecyl sulfate (SDS), 0.5M NaPO₄, 1 mM EDTA at 50° C. with washing in 1×SSC, 0.1% SDS at 50° C. or in 7% sodium dodecyl sulfate (SDS), 0.5M NaPO₄, 1 mM EDTA at 50° C. with washing in 0.5×SSC, 0.1% SDS at 50° C. In still further embodiments, the reference nucleotide sequence hybridizes to the “test” nucleotide sequence in 7% sodium dodecyl sulfate (SDS), 0.5M NaPO₄, 1 mM EDTA at 50° C. with washing in 0.1×SSC, 0.1% SDS at 50° C., or in 7% sodium dodecyl sulfate (SDS), 0.5M NaPO₄, 1 mM EDTA at 50° C. with washing in 0.1×SSC, 0.1% SDS at 65° C.

In some embodiments of the invention, nucleotide sequences having significant sequence identity to the nucleotide sequences of the invention (e.g., encoding PON degrading enzymes) are provided. “Significant sequence identity” or “significant sequence similarity” means at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, and/or 100% identity or similarity with another nucleotide sequence. Thus, in additional embodiments, “significant sequence identity” or “significant sequence similarity” means a range of about 70% to about 100%, about 75% to about 100%, about 80% to about 100%, about 81% to about 100%, about 82% to about 100%, about 83% to about 100%, about 84% to about 100%, about 85% to about 100%, about 86% to about 100%, about 87% to about 100%, about 88% to about 100%, about 89% to about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, and/or about 99% to about 100% identity or similarity with another nucleotide sequence. Therefore, in some embodiments, a nucleotide sequence of the invention is a nucleotide sequence that has significant sequence identity to the nucleotide sequence of any of SEQ ID NO:1 and/or SEQ ID NO:2. In particular embodiments, a nucleotide sequence of the invention is a nucleotide sequence that has at least 75% sequence identity to the nucleotide sequence of any of, for example, SEQ ID NO:1 and/or SEQ ID NO:2, and wherein said nucleotide sequence encodes a polypeptide comprises PON degrading activity.

In some embodiments, a polypeptide of the invention comprises, consists essentially of, or consists of an amino acid sequence that is at least 70% identical, e.g., at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, and/or 100% identical to an amino acid sequence of, for example, SEQ ID NO:3, and which comprises PON degrading activity.

In some embodiments, a polypeptide or nucleotide sequence of the invention can be a conservatively modified variant. As used herein, “conservatively modified variant” refer to polypeptide and nucleotide sequences containing individual substitutions, deletions or additions that alter, add or delete a single amino acid or nucleotide or a small percentage of amino acids or nucleotides in the sequence, where the alteration results in the substitution of an amino acid with a chemically similar amino acid. Conservative substitution tables providing functionally similar amino acids are well known in the art.

As used herein, a conservatively modified variant of a polypeptide is biologically active and therefore possesses the desired activity of the reference polypeptide (e.g., PON degrading activity; reducing PON and/or NNK content in a plant) as described herein. The variant can result from, for example, a genetic polymorphism or human manipulation. A biologically active variant of the reference polypeptide can have at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity or similarity (e.g., about 40% to about 99% or more sequence identity or similarity and any range therein) to the amino acid sequence for the reference polypeptide as determined by sequence alignment programs and parameters described elsewhere herein. An active variant can differ from the reference polypeptide sequence by as few as 1-15 amino acid residues, as few as 1-10, such as 6-10, as few as 5, as few as 4, 3, 2, or even 1 amino acid residue.

Naturally occurring variants may exist within a population. Such variants can be identified by using well-known molecular biology techniques, such as the polymerase chain reaction (PCR), and hybridization as described below. Synthetically derived nucleotide sequences, for example, sequences generated by site-directed mutagenesis or PCR-mediated mutagenesis which still encode a polypeptide of the invention, are also included as variants. One or more nucleotide or amino acid substitutions, additions, or deletions can be introduced into a nucleotide or amino acid sequence disclosed herein, such that the substitutions, additions, or deletions are introduced into the encoded protein. The additions (insertions) or deletions (truncations) may be made at the N-terminal or C-terminal end of the native protein, or at one or more sites in the native protein. Similarly, a substitution of one or more nucleotides or amino acids may be made at one or more sites in the native protein.

For example, conservative amino acid substitutions may be made at one or more predicted, preferably nonessential amino acid residues. A “nonessential” amino acid residue is a residue that can be altered from the wild-type sequence of a protein without altering the biological activity, whereas an “essential” amino acid is required for biological activity. A “conservative amino acid substitution” is one in which the amino acid residue is replaced with an amino acid residue with a similar side chain. Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif, where such residues are essential for protein activity.

For example, amino acid sequence variants of the reference polypeptide can be prepared by mutating the nucleotide sequence encoding the enzyme. The resulting mutants can be expressed recombinantly in plants, and screened for those that retain biological activity by assaying for increased or reduced nicotine content using standard assay techniques as described herein. Methods for mutagenesis and nucleotide sequence alterations are known in the art. See, e.g., Kunkel (1985) Proc. Natl. Acad. Sci. USA 82:488-492; Kunkel et al. (1987) Methods in Enzymol. 154:367-382; and Techniques in Molecular Biology (Walker & Gaastra eds., MacMillan Publishing Co. 1983) and the references cited therein; as well as U.S. Pat. No. 4,873,192. Clearly, the mutations made in the DNA encoding the variant must not disrupt the reading frame and preferably will not create complimentary regions that could produce secondary mRNA structure. See, EP Patent Application Publication No. 75,444. Guidance as to appropriate amino acid substitutions that do not affect biological activity of the protein of interest may be found in the model of Dayhoff et al. (1978) Atlas of Protein Sequence and Structure (National Biomedical Research Foundation, Washington, D.C.), herein incorporated by reference.

The deletions, insertions and substitutions in the polypeptides described herein are not expected to produce radical changes in the characteristics of the polypeptide (e.g., the activity of the polypeptide). However, when it is difficult to predict the exact effect of the substitution, deletion or insertion in advance of doing so, one of skill in the art will appreciate that the effect can be evaluated by routine screening assays that can screen for the particular polypeptide activities of interest (e.g., reduced PON content).

In some embodiments, the compositions of the invention can comprise active fragments of the polypeptide. As used herein, “fragment” means a portion of the reference polypeptide that retains the polypeptide activity of conferring increased or decreased nicotine content in a plant. A fragment also means a portion of a nucleic acid molecule encoding the reference polypeptide. An active fragment of the polypeptide can be prepared, for example, by isolating a portion of a polypeptide-encoding nucleic acid molecule that is expressed to produce the encoded fragment of the polypeptide (e.g., by recombinant expression in vitro), and assessing the activity of the fragment. Nucleic acid molecules encoding such fragments can be at least about 150, 200, 250, 300, 350, 400, 450, 500, 550, 600, 650, 700, 800, 900, 1,000, 1,100, 1,200, 1,300, 1,400, 1,500 contiguous nucleotides, or up to the number of nucleotides present in a full-length polypeptide-encoding nucleic acid molecule. As such, polypeptide fragments can be at least about 50, 60, 70, 80, 90, 100, 125, 150, 175, 200, 225, 250, 275, 300, 325, 350, 375, 400, 425, 450, 475, 500 contiguous amino acid residues, or up to the total number of amino acid residues present in the full-length polypeptide.

Thus, in some embodiments, a variant or functional fragment of a polypeptide of this invention or a variant or functional fragment having substantial identity to a polypeptide sequence of this invention (e.g., SEQ ID NO:2, SEQ ID NO:7) when produced in a transgenic plant reduces PON content of the transgenic plant producing said polypeptides, thereby reducing the amount of PON and/or NNK in a tobacco product produced from said transgenic plant.

In some embodiments, the nucleotide sequences and/or nucleic acid molecules of the invention can be operably/operatively linked to a variety of promoters for expression in host cells (e.g., plant cells). Thus, in some embodiments, the invention provides transformed host cells and transformed organisms comprising the transformed host cells, wherein the host cells and organisms are transformed with one or more nucleic acid molecules/nucleotide sequences of the invention. As used herein, “operably linked to,” when referring to a first nucleic acid sequence that is operably linked to a second nucleic acid sequence, means a situation when the first nucleic acid sequence is placed in a functional relationship with the second nucleic acid sequence. For instance, a promoter is operably associated with a coding sequence if the promoter effects the transcription or expression of the coding sequence.

In the context of a polypeptide, “operably linked to,” when referring to a first polypeptide sequence that is operably linked to a second polypeptide sequence, means a situation when the first polypeptide sequence is placed in a functional relationship with the second polypeptide sequence. For instance, a polypeptide of interest (e.g., a PON degrading enzyme) can be operably linked to a targeting sequence (e.g., vacuolar targeting sequence or ER targeting signal sequence) if the targeting sequence effects the targeting/localization of the polypeptide sequence of interest in a cell or an organism.

Any promoter useful for initiation of transcription in a cell of a plant can be used in the expression cassettes of the present invention. A “promoter,” as used herein, is a nucleotide sequence that controls or regulates the transcription of a nucleotide sequence (i.e., a coding sequence) that is operably associated with the promoter. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters can include, for example, constitutive, inducible, temporally regulated, developmentally regulated, chemically regulated, tissue-preferred and tissue-specific promoters for use in the preparation of recombinant nucleic acid molecules, i.e., chimeric genes.

The choice of promoter will vary depending on the temporal and spatial requirements for expression, and also depending on the host cell to be transformed. Thus, for example, where expression in a specific tissue or organ is desired, a tissue-specific or tissue preferred promoter can be used (e.g., a root or leaf specific/preferred promoter). In contrast, where expression in response to a stimulus is desired a promoter inducible by stimuli or chemicals can be used. Where continuous expression at a relatively constant level is desired throughout the cells or tissues of an organism a constitutive promoter can be chosen.

Thus, promoters useful with the invention include, but are not limited to, those that drive expression of a nucleotide sequence constitutively, those that drive expression when induced, and those that drive expression in a tissue- or developmentally-specific manner. These various types of promoters are known in the art. Further, promoters can be identified in and isolated from a plant to be transformed and then inserted into an expression cassette to be used in transformation of said plant or another plant.

Examples of constitutive promoters include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter obtained from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). In representative embodiments, the nucleotide sequences of this invention (e.g., nucleotide sequence encoding PON degrading enzymes) can be operably linked to a constitutive promoter as described herein.

In some embodiments, tissue specific/tissue preferred promoters can be used. Tissue specific or preferred expression patterns include, but are not limited to, green tissue specific or preferred, root specific or preferred, stem specific or preferred, and flower specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants include, but are not limited to, those that direct expression in root, pith, leaf or pollen. Such promoters are disclosed, for example, in WO 93/07278, herein incorporated by reference in its entirety for the teachings relevant to this sentence and/or paragraph. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; and the cestrum yellow leaf curling virus promoter disclosed in WO 01/73087, all incorporated by reference in their entireties for the teachings relevant to this sentence and/or paragraph.

Additional examples of tissue-specific/tissue preferred promoters include, but are not limited to, the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612). In some particular embodiments, the nucleotide sequences of the invention are operatively associated with a root-preferred promoter.

Particularly useful for seed-specific expression is the pea vicilin promoter (Czako et al. (1992) Mol. Gen. Genet. 235:33-40; as well as the seed-specific promoters disclosed in U.S. Pat. No. 5,625,136.

Useful promoters for expression in mature leaves are those that are switched on at the onset of senescence, such as the SAG promoter from Arabidopsis (Gan et al. (1995) Science 270:1986-1988). In representative embodiments, a senescence inducible promoter can be operably linked to the nucleotide sequences encoding the PON degrading enzymes. Additional examples of a senescence-inducible promoter is the promoter from the CYP82E4 gene (Chakrabarti et al. Plant Mol. Biol. 66: 415-427 (2008). The promoter of the CYP82E4 gene is not only activated during natural senescence, but is also very active during air-curing, which is a form of controlled senesence. Since TSNA formation is greatly enhanced during curing, it may be advantageous to have the nucleotide sequence of this invention (e.g., nucleotide sequence encoding PON degrading enzymes) expressed during this time.

In addition, promoters functional in plastids can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

In some embodiments of the invention, inducible promoters can be used. Thus, for example, chemical-regulated promoters can be used to modulate the expression of a gene in a plant through the application of an exogenous chemical regulator. Regulation of the expression of nucleotide sequences of the invention via promoters that are chemically regulated enables the polypeptides of the invention to be synthesized only when the crop plants are treated with the inducing chemicals. Depending upon the objective, the promoter may be a chemical-inducible promoter, where application of a chemical induces gene expression, or a chemical-repressible promoter, where application of the chemical represses gene expression.

Chemical inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1 a promoter, which is activated by salicylic acid (e.g., the PR1a system), steroid steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257) and tetracycline-inducible and tetracycline-repressible promoters (see, e.g., Gatz et al. (1991) Mol. Gen. Genet. 227, 229-237, and U.S. Pat. Nos. 5,814,618 and 5,789,156, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters.

Other non-limiting examples of inducible promoters include ABA- and turgor-inducible promoters, the auxin-binding protein gene promoter (Schwob et al. (1993) Plant J 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), and the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Int'l Patent Application Publication Nos. WO 97/06269 and WO 97/06268) systems and glutathione S-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108. Other chemically inducible promoters useful for directing the expression of the nucleotide sequences of this invention in plants are disclosed in U.S. Pat. No. 5,614,395 herein incorporated by reference in its entirety. Chemical induction of gene expression is also detailed in the published application EP 0 332 104 (to Ciba-Geigy) and U.S. Pat. No. 5,614,395. In some embodiments, a promoter for chemical induction can be the tobacco PR-1a promoter.

As used herein, “expression cassette” means a nucleic acid molecule comprising a nucleotide sequence of interest (e.g., a nucleotide sequence encoding a PON degrading enzyme), wherein said nucleotide sequence is operatively associated with at least a control sequence (e.g., a promoter). Thus, some embodiments of the invention provide expression cassettes designed to express the nucleotides sequences of the invention. In this manner, for example, one or more plant promoters operatively associated with one or more nucleotide sequences encoding PON degrading enzymes (e.g., SEQ ID NO:1, SEQ ID NO:2, and/or SEQ ID NO:6, and/or a nucleotide sequence encoding one or more polypeptides having the amino acid sequences of SEQ ID NO:3 or SEQ ID NO:7) are provided in expression cassettes for expression in an organism or cell thereof (e.g., a plant, plant part and/or plant cell).

An expression cassette comprising a nucleotide sequence of interest may be chimeric, meaning that at least one of its components is heterologous with respect to at least one of its other components. An expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event.

In addition to the promoters operably linked to the nucleotide sequences of the invention, an expression cassette of the invention can also include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, promoters, enhancers, introns, 5′ and 3′ untranslated regions, translation leader sequences, termination signals, and polyadenylation signal sequences.

For purposes of the invention, the regulatory sequences or regions can be native/analogous to the plant, plant part and/or plant cell and/or the regulatory sequences can be native/analogous to the other regulatory sequences. Alternatively, the regulatory sequences may be heterologous to the plant (and/or plant part and/or plant cell) and/or to each other (i.e., the regulatory sequences). Thus, for example, a promoter can be heterologous when it is operatively linked to a polynucleotide from a species different from the species from which the polynucleotide was obtained. Alternatively, a promoter can also be heterologous to a selected nucleotide sequence if the promoter is from the same/analogous species from which the polynucleotide is obtained, but one or both (i.e., promoter and/or polynucleotide) are substantially modified from their original form and/or genomic locus, and/or the promoter is not the native promoter for the operably linked polynucleotide.

A number of non-translated leader sequences obtained from viruses are known to enhance gene expression. Specifically, leader sequences from Tobacco Mosaic Virus (TMV, the “co-sequence”), Maize Chlorotic Mottle Virus (MCMV) and Alfalfa Mosaic Virus (AMV) have been shown to be effective in enhancing expression (Gallie et al. (1987) Nucleic Acids Res. 15:8693-8711; and Skuzeski et al. (1990) Plant Mol. Biol. 15:65-79). Other leader sequences known in the art include, but are not limited to, picornavirus leaders such as an encephalomyocarditis (EMCV) 5′ noncoding region leader (Elroy-Stein et al. (1989) Proc. Natl. Acad. Sci. USA 86:6126-6130); potyvirus leaders such as a Tobacco Etch Virus (TEV) leader (Allison et al. (1986) Virology 154:9-20); Maize Dwarf Mosaic Virus (MDMV) leader (Allison et al. (1986), supra); human immunoglobulin heavy-chain binding protein (BiP) leader (Macejak & Samow (1991) Nature 353:90-94); untranslated leader from the coat protein mRNA of AMV (AMV RNA 4; Jobling & Gehrke (1987) Nature 325:622-625); tobacco mosaic TMV leader (Gallie et al. (1989) Molecular Biology of RNA 237-256); and MCMV leader (Lommel et al. (1991) Virology 81:382-385). See also, Della-Cioppa et al. (1987) Plant Physiol. 84:965-968.

An expression cassette also can optionally include a transcriptional and/or translational termination region (i.e., termination region) that is functional in plants. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the plant host, or may be obtained from another source (i.e., foreign or heterologous to the promoter, the nucleotide sequence of interest, the plant host, or any combination thereof). Appropriate transcriptional terminators include, but are not limited to, the CAMV 35S terminator, the tml terminator, the nopaline synthase terminator and/or the pea rbcs E9 terminator.

An expression cassette of the invention also can include a nucleotide sequence for a selectable marker, which can be used to select a transformed plant, plant part and/or plant cell. As used herein, “selectable marker” means a nucleotide sequence that when expressed imparts a distinct phenotype to the plant, plant part and/or plant cell expressing the marker and thus allows such transformed plants, plant parts and/or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening (e.g., the R-locus trait). Of course, many examples of suitable selectable markers are known in the art and can be used in the expression cassettes described herein.

Examples of selectable markers include, but are not limited to, a nucleotide sequence encoding neo or nptII, which confers resistance to kanamycin, G418, and the like (Potrykus et al. (1985) Mol. Gen. Genet. 199:183-188); a nucleotide sequence encoding bar, which confers resistance to phosphinothricin; a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin. One of skill in the art is capable of choosing a suitable selectable marker for use in an expression cassette of the invention.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac,” pp. 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J. Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding aequorin, which may be employed in calcium-sensitive bioluminescence detection (Prasher et al. (1985) Biochem. Biophys. Res. Comm. 126:1259-1268); or a nucleotide sequence encoding green fluorescent protein (Niedz et al. (1995) Plant Cell Reports 14:403-406). One of skill in the art is capable of choosing a suitable selectable marker for use in an expression cassette of the invention.

An expression cassette of the invention also can include nucleotide sequences that encode other desired traits. Such desired traits can be other nucleotide sequences which confer various agriculturally desirable traits such as disease and/or insect resistance, herbicide resistance, abiotic stress tolerance or resistance and the like. Such nucleotide sequences can be stacked with any combination of nucleotide sequences to create plants, plant parts or plant cells having the desired phenotype. Stacked combinations can be created by any method including, but not limited to, cross breeding plants by any conventional methodology, or by genetic transformation. If stacked by genetically transforming the plants, nucleotide sequences encoding additional desired traits can be combined at any time and in any order. For example, a transgenic plant comprising one or more desired traits can be used as the target to introduce further traits by subsequent transformation. The additional nucleotide sequences can be introduced simultaneously in a co-transformation protocol with a nucleotide sequence, nucleic acid molecule, nucleic acid construct, and/or composition of the invention, provided by any combination of expression cassettes. For example, if two nucleotide sequences will be introduced, they can be incorporated in separate cassettes (trans) or can be incorporated on the same cassette (cis). Expression of the nucleotide sequences can be driven by the same promoter or by different promoters. It is further recognized that nucleotide sequences can be stacked at a desired genomic location using a site-specific recombination system. See, e.g., Int'l Patent Application Publication Nos. WO 99/25821; WO 99/25854; WO 99/25840; WO 99/25855 and WO 99/25853.

In addition to expression cassettes, the nucleic acid molecules and nucleotide sequences described herein can be used in connection with vectors. The term “vector” refers to a composition for transferring, delivering or introducing a nucleic acid (or nucleic acids) into a cell. A vector comprises a nucleic acid molecule comprising the nucleotide sequence(s) to be transferred, delivered or introduced. Vectors for use in transformation of plants and other organisms are well known in the art. Non-limiting examples of general classes of vectors include, but are not limited to, a viral vector, a plasmid vector, a phage vector, a phagemid vector, a cosmid, a fosmid, a bacteriophage, or an artificial chromosome. The selection of a vector will depend upon the preferred transformation technique and the target species for transformation. Accordingly, in further embodiments, a recombinant nucleic acid molecule of the invention can be comprised within a recombinant vector. The size of a vector can vary considerably depending on whether the vector comprises one or multiple expression cassettes (e.g., for molecular stacking). Thus, a vector size can range from about 3 kb to about 30 kb. Thus, in some embodiments, a vector is about 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 40 kb, 50 kb, 60 kb, and the like or any range therein, in size. In some particular embodiments, a vector can be about 3 kb to about 15 kb in size.

The present invention is directed in part to the discovery that expressing in a plant at least one isolated nucleic acid molecule or nucleic acid construct of this invention comprising a nucleotide sequence encoding a PON degrading enzyme can result in the plant having reduced PON and/or NNK content as compared to a plant that does not comprise said isolate nucleic acid molecule or nucleic acid construct. Thus, in some embodiments of the invention, a method of producing a transgenic plant cell is provided, said method comprising introducing into a plant cell an isolated nucleic acid molecule/construct of the invention, thereby producing a transgenic plant cell that can regenerate a transgenic plant having decreased PON and/or NNK content as compared to a plant regenerated from a plant cell that does not comprise said nucleic acid molecule/construct. In some embodiments, the transgenic plant cell comprises more than one (e.g., 2, 3, 4, 5, 6, 7, 8, 9, 10, etc.) nucleic acid molecule/nucleotide sequence of the invention. Thus, in some aspects of the invention, the transgenic plants, or parts thereof, comprise and express one or more isolated nucleic acid molecule/constructs of the invention, thereby producing one or more polypeptides of the invention resulting in reduced PON and/or NNK content in said plant cell and regenerated transgenic plant, wherein tobacco products obtained from said regenerated transgenic tobacco plant have reduced PON and/or reduced NNK content.

“Introducing,” in the context of a nucleotide sequence of interest (e.g., the nucleic acid molecules/constructs/expression cassettes of the invention), means presenting the nucleotide sequence of interest to the plant, plant part, and/or plant cell in such a manner that the nucleotide sequence gains access to the interior of a cell. Where more than one nucleotide sequence is to be introduced these nucleotide sequences can be assembled as part of a single polynucleotide or nucleic acid construct, or as separate polynucleotide or nucleic acid constructs, and can be located on the same or different transformation vectors. Accordingly, these polynucleotides can be introduced into plant cells in a single transformation event, in separate transformation events, or, e.g., as part of a breeding protocol. Thus, the term “transformation” as used herein refers to the introduction of a heterologous nucleic acid into a cell. Transformation of a cell may be stable or transient. Thus, in some embodiments, a plant, plant part or plant cell of the invention is stably transformed with a nucleic acid molecule of the invention. In other embodiments, a plant, plant part or plant cell of the invention is transiently transformed with a nucleic acid molecule of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended the introduced polynucleotide is stably incorporated into the genome of the cell, and thus the cell is stably transformed with the polynucleotide.

“Stable transformation” or “stably transformed” as used herein means that a nucleic acid is introduced into a cell and integrates into the genome of the cell. As such, the integrated nucleic acid is capable of being inherited by the progeny thereof, more particularly, by the progeny of multiple successive generations. “Genome” as used herein also includes the nuclear and the plastid genome, and therefore includes integration of the nucleic acid into, for example, the chloroplast genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

A nucleic acid molecule of the invention (e.g., comprising one or more of the nucleotide sequences of SEQ ID NO:1, SEQ ID NO:2, SEQ ID NO:6 or a nucleotide sequence encoding one or more polypeptides having the amino acid sequence of any one of SEQ ID NO:3, SEQ ID NO:7) can be introduced into a cell by any method known to those of skill in the art.

In some embodiments of the invention, transformation of a cell comprises nuclear transformation. In other embodiments, transformation of a cell comprises plastid transformation (e.g., chloroplast transformation).

Procedures for transforming plants are well known and routine in the art and are described throughout the literature. Non-limiting examples of methods for transformation of plants include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Agrobacterium-mediated transformation is a commonly used method for transforming plants, in particular, dicot plants, because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Höfgen & Willmitzer (1988) Nucleic Acids Res. 16:9877).

Transformation of a plant by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

Another method for transforming plants, plant parts and/or plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacterium or a bacteriophage, each containing one or more nucleic acids sought to be introduced) also can be propelled into plant tissue.

Thus, in particular embodiments of the invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

Likewise, the genetic properties engineered into the transgenic seeds, plants, plant parts, and/or plant cells of the invention described above can be passed on by sexual reproduction or vegetative growth and therefore can be maintained and propagated in progeny plants. Generally, maintenance and propagation make use of known agricultural methods developed to fit specific purposes such as harvesting, sowing or tilling.

A nucleotide sequence therefore can be introduced into the tobacco plant, plant part and/or plant cell in any number of ways that are well known in the art. The methods of the invention do not depend on a particular method for introducing one or more nucleotide sequences into a plant, only that they gain access to the interior of at least one cell of the plant. Where more than one nucleotide sequence is to be introduced, they can be assembled as part of a single nucleic acid construct, or as separate nucleic acid constructs, and can be located on the same or different nucleic acid constructs. Accordingly, the nucleotide sequences can be introduced into the cell of interest in a single transformation event, in separate transformation events, or, for example, in plants, as part of a breeding protocol.

Thus, in additional embodiments, the invention provides a method of producing a tobacco plant, plant part and/or plant cell having reduced PON content and/or reduced NNK content, the method comprising introducing into said tobacco plant, plant part and/or plant cell one or more nucleic acid molecules of the invention to produce a transgenic tobacco plant, plant part and/or plant cell, wherein the transgenic tobacco plant tobacco plant, plant part and/or plant cell comprises said nucleic acid construct of the invention in its genome, thereby producing a tobacco plant, plant part and/or plant cell having reduced PON content and/or reduced NNK content as compared to a control tobacco plant, plant part and/or plant cell that does not comprise said nucleic acid construct.

In some embodiments, the invention provides a method of producing a tobacco plant, plant part and/or plant cell having reduced PON content and/or reduced NNK content, the method comprising introducing into a tobacco plant cell a nucleic acid molecule of the invention to produce a transgenic tobacco plant cell, wherein the transgenic tobacco plant cell comprises said nucleic acid construct of the invention in its genome; and regenerating said transgenic tobacco plant cell into a tobacco plant and/or plant part to produce a transgenic tobacco plant and/or plant part comprising said nucleic acid construct, thereby producing a tobacco plant and/or plant part having reduced PON content and/or reduced NNK content as compared to a control tobacco plant and/or plant part that does not comprise said nucleic acid construct.

In some embodiments, the present invention provides a method of reducing PON and/or NNK content in a tobacco plant, plant part and/or plant cell, comprising introducing into a tobacco plant, plant part and/or plant cell one or more nucleic acid constructs of the invention to produce a transgenic tobacco plant, plant part and/or plant cell comprising said nucleic acid construct, thereby reducing PON and/or NNK content in said transgenic tobacco plant, plant part and/or plant cell as compared to a control tobacco plant, plant part and/or plant cell that is not transformed with the said nucleic acid construct.

In an additional embodiment, a method of reducing PON and/or NNK content in a tobacco plant, plant part and/or plant cell is provided, comprising introducing into a tobacco plant cell a nucleic acid construct of the invention to produce a transgenic tobacco plant cell comprising said nucleic acid construct; and regenerating said transgenic tobacco plant cell to produce a transgenic tobacco plant comprising said nucleic acid construct, thereby reducing PON and/or NNK content in said transgenic tobacco plant as compared to a control tobacco plant that is not transformed with the said nucleic acid construct.

In still further aspects, the present invention provides a method of producing a tobacco product having reduced PON and/or NNK content, the method comprising: introducing into a tobacco plant, plant part and/or plant cell one or more heterologous nucleic acid molecules comprising a nucleotide sequence encoding a PON degrading enzyme, thereby producing a transgenic tobacco plant, plant part and/or plant cell having reduced PON and/or NNK content as compared to a tobacco plant, plant part and/or plant cell not transformed with said one or more heterologous nucleic acid molecules; and producing a tobacco product from said transgenic tobacco plant, plant part and/or plant cell, wherein the tobacco product has reduced PON and/or NNK content as compared to a tobacco product produced from a plant, plant part and/or plant cell not transformed with said one or more heterologous nucleic acid molecules.

Procedures for determining PON content and NNK content are well known and routine in the art and are described throughout the literature. Non-limiting examples of methods for measuring PON content include such methods as those provided in Wei (Studies on the biosynthesis and metabolites of pyridine alkaloids in Nicotiana species. Ph.D. Thesis. Univ. of Kentucky (2000)). Other methods are described in the “legacy documents” at legacy.library.ucsf.edu/tid/zcx53d00/pdf. Hecht et al. provides a further method of analyzing PON (referred to as aminoketone) (Proc. Natl. Acad. Sci. 97(23):12493-12497 (2000)).

Methods for measuring TSNA content, including NNK, are well known in the art and include assays based on, for example, gas chromatography-mass spectrometry and others based on liquid chromatography-mass spectrometry (see, for example, Lewis et al. (Plant Biotech. J. 6:346-354 (2008)).

A further aspect of the invention provides transformed non-human host cells and transformed non-human organisms comprising the transformed non-human cells, wherein the transformed cells and transformed organisms comprise nucleic acid molecules comprising one or more heterologous nucleic acid molecules and/or nucleotide sequences of the invention (e.g., encoding PON degrading enzymes). In some embodiments, the transformed non-human host cell includes but is not limited to a transformed bacterial cell, and/or a transformed plant cell. Thus, in some embodiments, the transformed non-human organism comprising the transformed non-human host cell includes, but is not limited to, a transformed bacterium, and/or a transformed plant.

In some particular embodiments, the invention provides a transgenic plant cell comprising a nucleic acid molecule of the invention and/or a transgenic plant regenerated from said transgenic plant cell. Accordingly, in some embodiments of the invention, a transgenic plant having reduced PON and/or NNK content is provided, said transgenic plant regenerated from a transgenic plant cell comprising at least one isolated nucleic acid molecule/nucleic acid construct of the invention.

Additional aspects of the invention include a harvested product produced from the transgenic plants and/or parts thereof of the invention, as well as a processed product (e.g., tobacco product) produced from said harvested product. A harvested product can be a whole plant or any plant part, wherein said harvested product comprises a recombinant nucleic acid molecule/construct of the invention. Thus, in some embodiments, non-limiting examples of a harvested product include a seed, a fruit, a flower or part thereof (e.g., an anther, a stigma, and the like), a leaf, a stem, an extract from said plants and/or plant parts, and the like.

“Tobacco product” refers to a product comprising material produced by a Nicotiana plant. In some embodiments, a tobacco product includes, but is not limited to, a cured tobacco leaf produced from a tobacco plant of this invention (e.g., a tobacco plant comprising a nucleotide sequence encoding a PON degrading enzyme). Non-limiting examples of different types of cured leaf include flue-cured, air-cured, fire-cured and sun-cured. In other embodiments, a tobacco product can be a fermented tobacco product. Fermented tobacco products include, but are not limited to those used in smokeless products (e.g., lozenges, patches, gum, electronic cigarettes, and the like).

Additional non-limiting examples of tobacco products include nicotine gum and patches for smoking cessation, cigarette tobacco including expanded (puffed) and reconstituted tobacco, cigar tobacco, pipe tobacco, cigarettes, cigars, and all forms of smokeless tobacco such as chewing tobacco, snuff, snus and lozenges. “Cigarettes” includes electronic cigarettes and “heat not burn” products which are cigarette-like devices that heat tobacco rather than burn tobacco.

Thus, in additional embodiments, the present invention provides a tobacco product produced from the transgenic tobacco plants, plant parts, plant cells, and/or the progeny plants thereof, wherein the product has a reduced amount of PON and/or NNK. A tobacco product can be any product made using the tobacco plants, plant parts or plant cells of this invention. Accordingly a tobacco product can be leaf tobacco, shredded tobacco, cut tobacco, ground tobacco, powder tobacco, tobacco extract, smokeless tobacco, moist or dry snuff, kretek, pipe tobacco, cigar tobacco, cigarillo tobacco, cigarette tobacco, chewing tobacco, bidis, bits, and tobacco-containing gum, lozenges, or any combination thereof. In other embodiments, the tobacco product can be a cigarette (including an electronic cigarette), cigarillo, a non-ventilated recess filter cigarette, a vented recess filter cigarette, a cigar, snuff, chewing tobacco, or any combination thereof. In some embodiments, the tobacco extract made from the transgenic tobacco plants, plant parts or plant cells of this invention, including nicotine purified from such extracts, can be used in electronic cigarettes.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1. Generation of PAO Constructs

The Pseudomonas HZN6 PAO nucleotide sequence was custom synthesized (GenScript, Piscataway, N.J.) in a manner optimized for expression in tobacco. For example, nucleotide substitutions were made throughout the gene to optimize for preferred codons in tobacco, eliminate cryptic splice sites and premature polyA sites, optimize overall GC content and eliminate RNA instability motifs. In addition, given that regulatory and/or mRNA stability motifs found in 5′ and 3′ untranslated regions (UTRs) differ greatly between prokaryotes and eukaryotes, the PAO sequence was also engineered to contain 5′ and 3′ UTR sequences from the tobacco CYP82E10 gene (Lewis et al., Phytochemistry 71:1988-1998 (2010)) in order to further enhance transcript stability and function. The optimized PAO nucleotide sequence was cloned into plant expression vector pBI121 by substituting the GUS reporter gene within this vector with the optimized PAO nucleotide sequence, thereby placing it under the transcriptional control of the strong constitutive 35S promoter of Cauliflower Mosaic Virus (CaMV), with transcript termination and polyadenylation mediated by the nos termination motif originating from nopaline synthase gene of Agrobacterium tumefaciens (Chen et al. Mol. Breed. 11:287-293 (2003)). The design of this construct, designated 35S:PAO, is expected to facilitate the synthesis of the PAO enzyme within the cytosol of the cell.

Although the intracellular localization of PON is unknown, the major tobacco alkaloid, nicotine, is primarily stored within the large central vacuole. If PON is predominantly sequestered in the vacuole, then targeting PAO to this organelle may be preferred over cytosolic localization of the enzyme. To mediate the translocation of PAO to the vacuole, an additional construct was made in which the sequences encoding the first 50 amino acids of BBLa were fused at the 5′ end of PAO. BBLa is vacuolar localized berberine bridge-like enzyme involved in a late stage of tobacco alkaloid biosynthesis, but whose catalytic properties have yet to be defined (Kajikawa et al. Plant Physiol. 155:2010-2022 (2011)). In this same report by Kajikawa et al., attachment of the first 50 amino acids of BBLa to the GFP reporter protein was shown to be sufficient for transporting GFP into the vacuole of tobacco cells. Sequences encoding the BBLa N-terminal/PAO fusion protein were cloned into pBI121 and the construct was designated 35S:BBL_{vac}-PAO. Similar to 35S:PAO, the 3′-UTR sequences of the 35S:BBL_{vac}-PAO construct originated from CYP82E10; the 5′-UTR, however, corresponds to that of the native BBLa gene. Finally, a full-length cDNA of BBLa was also cloned into pBI121 and designated 35S:BBLa. The nucleotide and predicted protein sequences of each of the constructs used in this study are shown in the Appendix.

### Example 2. Transformation

Constructs 35S:PAO, 35S:BBL_{vac}-PAO, and 35S:BBLa were transferred into Agrobacterium tumefaciens strain GV3101, which were then introduced into both a burley (TN90 SRC) and flue-cured (K326 SRC) tobacco cultivar using standard Agrobacterium-mediated transformation protocols (Horsch et al., 1985). As a vector control, these same tobacco lines were also transformed with a construct containing the GUS reporter gene under the transcriptional control of a senescence-inducible promoter within the same pBI121 vector backbone (E4:GUS). The T₀ generation transgenic plants were grown in soil to the 7-10 leaf stage (about 5 to 6 inches high) prior to assaying for their ability to metabolize PON.

### Example 3. PON Analysis

Young tobacco plants grown in a lab environment produce far less alkaloid than large field-grown plant under commercial cultivation. Therefore, detecting and quantitating endogenous PON in these materials required a different method than that used for commercially cultivated plants. Thus, a detached leaf assay was used as an alternative method for determining whether any of the introduced transgenes can produce enzymes capable of metabolizing PON when expressed within the environment of the tobacco cell. In the detached leaf assay, PON labeled with the stable isotope deuterium [4-(methyl-d3-amino)-1-(3-pyridyl)-1-butanone; Toronto Research Chemicals] was introduced into the leaf through the transpirational stream. Individual leaves, ranging from 1-4 g fresh weight, from various independent T₀ transgenic plants were excised at the base of the petiole with a razor blade and quickly transferred to a conical receptacle containing 1 ml of a 0.4 mM solution of the d₃-PON in 10 mM phosphate buffer (pH 7.4). The detached leaves were incubated at room temperature under light until most of the solution had been taken up (typically between 1.5-2.5 hr). Afterwards, 75 ml of phosphate buffer were added, followed by an additional 24 hr incubation period. Each leaf was subsequently dried at 65° C. overnight, ground to a fine powder and analyzed for d₃-PON according to the protocol of Wei (Studies on the biosynthesis and metabolites of pyridine alkaloids in Nicotiana species. Ph.D. Thesis. Univ. of Kentucky (2000)).

The results of the detached leaf assays are shown in Table 1, below. Assuming that the uptake of d₃-PON was 100% efficient, each leaf would have taken up 100 μg of d₃-PON. By the end of the experiment, the d₃-PON content of leaves from plants containing the control E4:GUS construct ranged from 14.0 to 48.5 μg d₃-PON, averaging 32.9 μg in the K326 SRC background and 20.2 μg in TN90 SRC. Similarly, leaves containing the 35S:BBLa construct retained between 17.9-45.0 μg of d₃-PON, averaging 28.5 μg in K326 SRC and 35.1 μg in TN90 SRC. Leaves possessing the 35S:PAO transgene exhibited a particularly broad range of d₃-PON levels, with four of the plants exhibiting d₃-PON levels similar to that of the vector control and 35S:BBLa plants (679/35S:PAO-#5, 679/35S:PAO-#3, 678/35S:PAO-#6 and 678/35S:PAO-#12), and the other six containing d₃-PON levels below that observed in the control plant with the lowest amount of d₃-PON. Across all the independent T₀ individuals, plants containing the 35S:PAO construct averaged 14.0 μg d₃-PON in K326 SRC and 10.6 μg d₃-PON in TN90 SRC. Surprisingly, leaves from all nine T₀ plants containing the 35S:BBL_{vac}-PAO construct possessed d₃-PON levels lower than the lowest control or 35S:BBLa plant, and 8 of the 9 leaves contained less than 0.7 μg of d₃-PON. In contrast, of the six 35S:PAO plants whose leaves contained less d₃-PON than the controls, only one of these possessed less than 0.7 μg d₃-PON (679/35S:PAO-#16). Overall, the amount of d₃-PON in leaves from the K326 SRC transgenics averaged 1.8 μg, while the average in TN90 SRC was only 0.2 μg. The results observed with the 35S:BBL_{vac}-PAO transgenic tobacco plants is particularly remarkable given that independent T₀ plants typically display a wide range of transgene transcription phenotypes, due to variations in factors such as genomic insertion sites (position effects) and transgene copy number. Our results suggest that the activity of the 35S:BBL_{vac}-PAO gene product is easily saturated and that high levels of expression of this construct are not required in order to be effective.

From these examples, it is clear that expressing the PAO gene of Pseudomonas strain HZN6 within the cellular environment of tobacco can lead to the degradation of PON in these plants. It is also apparent that the use of constructs designed to facilitate the vacuolar localization of PAO increases the efficiency of PAO-mediated PON metabolism. Without wishing to be limited by any particular theory of the invention, two plausible explanations of this observation include: (1), vacuolar-localized PAO places the enzyme in the immediate proximity of the PON, a compound presumably stored in this organelle; and/or (2), the stability of the PAO enzyme per se may be greater within the vacuole (e.g. less susceptible to proteolytic turnover) than when localized to the cytosol. Finally, given that PON can serve as the direct alkaloid precursor in the formation of NNK, coupled with the observation that green and cured tobacco leaves possess a sufficient amount of PON to account for the NNK found in the cured leaf (Wei, Studies on the biosynthesis and metabolites of pyridine alkaloids in Nicotiana species. Ph.D. Thesis. Univ. of Kentucky (2000)), the transgenic plants expressing PAO described here should demonstrate a substantially reduced NNK phenotype in the cured leaf compared to conventional tobacco plants.

### Example 4. Analysis of T₀ Transgenic Plants

Additional T₀ individuals in the K326 SRC background were transplanted to the field to establish that the expression of constructs encoding PAO can mediate a decrease in the formation of NNK in the cured tobacco leaf. In addition to the constructs shown in Table 1, whose transgene expression was driven by the 35S CaMV promoter, transgenic plants expressing the PAO and BBL_{vac}-PAO constructs under the transcriptional control of the CYP82E4 (E4) promoter were also included in this study. The E4 promoter is strongly induced both during natural senescence and curing, and thus may have the potential of mediating higher levels of transgene expression more closely to the time when TSNA formation is occurring than may be possible using the 35S promoter (Chakrabarti et al, 2008 Plant Mol. Biol. 66: 415-427). All constructs were represented by at least six individual T₀ plants. Plants transformed with the E4:GUS reporter represented the controls. The field grown plants were fertilized and topped according to standard industry practice. At maturity, two leaves from the upper mid-stalk position of each plant were excised and flue-cured for about 3 days. Due to the unpredictability and variability of TSNA formation during the curing process, NOx gases were supplemented to the curing chamber during the curing period to facilitate TSNA production.

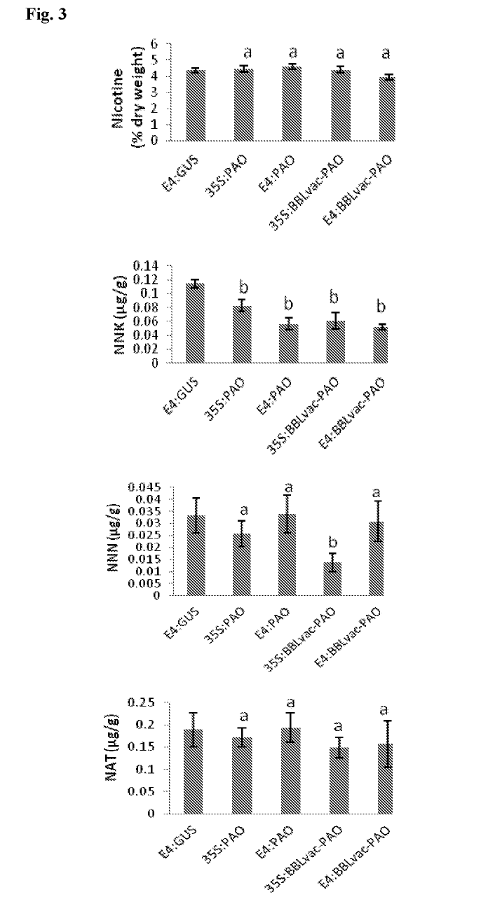

The results of both nicotine and TSNA analyses from the field study are shown in FIG. 3. NNK concentrations were significantly lower (P<0.05) in each of the genotypic groups possessing a PAO construct, in comparison to the control genotype. E4:GUS control plants averaged 0.115 μg/g NNK, whereas genotypes containing a PAO gene averaged between 0.052-0.083 μg/g NNK, representing a 55%-28% range in NNK reduction from that observed in the control plants. In contrast, there were no statistically significant differences in nicotine or NAT content between the E4:GUS control genotype and any of the genotypic groups containing a PAO construct. NNN concentration was also similar among all genotypes except for the plants containing the 35 S:BBL_{vac}-PAO construct. For reasons that are unclear, NNN was unexpectedly low in this group. NAB measurements are not presented because they were just at or below the level of detection in these samples. Cumulatively, the results shown in FIG. 3 suggest that the PAO transgene is capable of mediating specific reductions in NNK formation in the cured leaf.

### Example 5. Generation of Tobacco Plants Targeting Extracellular Matrix-Bound Pools of PON

The detached leaf assay results shown in Table 1 provide strong evidence that expression of PAO in the cytosol and/or vacuole is capable of metabolizing d₃-PON that was supplied exogenously through the transpirational stream. Furthermore, the results from the field study (FIG. 3) suggest that the expression of PAO in this manner can lead to reductions in NNK in flue-cured leaves.

TABLE 1
Quantitation of d3-PON in transgenic leaves fed one ml
of a 0.4 mM solution of d3-PON and incubated for 24 hr.

| PON T0 plant | | Concentration of | Total leaf dry | Total d3- | |
|---|---|---|---|---|---|
| (ug) | Cultivar | d3-PON (ppm)* | weight (g) | in leaf | |
| 679/E4:GUS-#11 | K326 SRC | 147.2 | 0.0953 | 14.0 | |
| 679/E4:GUS-#10 | K326 SRC | 433.6 | 0.0723 | 31.3 | |
| 679/E4:GUS-#11 | K326 SRC | 459.3 | 0.1055 | 48.5 | |
| 679/E4:GUS-#10 | K326 SRC | 276.1 | 0.1386 | 38.3 | |
| 679/E4:GUS-#5 | K326 SRC | 187.2 | 0.1727 | 32.3 | ave. = 32.9 |
| 679/35S:PAO-#16 | K326 SRC | 2.0 | 0.1242 | 0.3 | |
| 679/35S:PAO-#5 | K326 SRC | 257.9 | 0.1387 | 35.8 | |
| 679/35S:PAO-#4 | K326 SRC | 27.2 | 0.2213 | 6.0 | |
| 679/35S:PAO-#3 | K326 SRC | 145.2 | 0.1581 | 23.0 | |
| 679/35S:PAO-#9 | K326 SRC | 48.2 | 0.0974 | 4.7 | ave. = 14.0 |
| 679/35S:BBLa-#4 | K326 SRC | 111.1 | 0.1607 | 17.9 | |
| 679/35S:BBLa-#9 | K326 SRC | 112.2 | 0.2060 | 23.1 | |
| 679/35S:BBLa-#10 | K326 SRC | 102.7 | 0.2646 | 27.2 | |
| 679/35S:BBLa-#11 | K326 SRC | 135.3 | 0.2172 | 29.4 | |
| 679/35S:BBLa-#14 | K326 SRC | 256.7 | 0.1752 | 45.0 | ave. = 28.5 |
| 679/35S:BBLvac-PAO-#5 | K326 SRC | 2.2 | 0.1380 | 0.3 | |
| 679/35S:BBLvac-PAO-#1 | K326 SRC | 1.7 | 0.3388 | 0.6 | |
| 679/35S:BBLvac-PAO-#13 | K326 SRC | 1.6 | 0.1175 | 0.2 | |
| 679/35S:BBLvac-PAO-#12 | K326 SRC | 38.0 | 0.1620 | 6.2 | ave. = 1.8 |
| 678/E4:GUS-#5 | TN90 SRC | 161.6 | 0.1247 | 20.2 | |
| 678/E4:GUS-#4 | TN90 SRC | 90.0 | 0.2499 | 22.5 | |
| 678/E4:GUS-#2 | TN90 SRC | 93.8 | 0.1860 | 17.4 | |
| 678/E4:GUS-#16 | TN90 SRC | 83.6 | 0.2685 | 22.4 | |
| 678/E4:GUS-#3 | TN90 SRC | 65.1 | 0.2853 | 18.6 | ave. = 20.2 |
| 678/35S:PAO-#6 | TN90 SRC | 128.9 | 0.1878 | 24.2 | |
| 678/35S:PAO-#11 | TN90 SRC | 6.5 | 0.2403 | 1.6 | |
| 678/35S:PAO-#12 | TN90 SRC | 182.1 | 0.0851 | 15.5 | |
| 678/35S:PAO-#3 | TN90 SRC | 14.7 | 0.1522 | 2.2 | |
| 678/35S:PAO-#9 | TN90 SRC | 127.8 | 0.0735 | 9.4 | ave. = 10.6 |
| 678/35S:BBLa-#5 | TN90 SRC | 177.7 | 0.2023 | 36.0 | |
| 678/35S:BBLa-#7 | TN90 SRC | 174.6 | 0.1806 | 31.5 | |
| 678/35S:BBLa-#2 | TN90 SRC | 153.5 | 0.2407 | 36.9 | |
| 678/35S:BBLa-#15 | TN90 SRC | 208.4 | 0.1652 | 34.4 | |
| 678/35S:BBLa-#2 | TN90 SRC | 236.5 | 0.1542 | 36.5 | ave. = 35.1 |
| 678/35S:BBLvac-PAO-#14 | TN90 SRC | 0.8 | 0.2753 | 0.2 | |
| 678/35S:BBLvac-PAO-#3 | TN90 SRC | 0.8 | 0.1857 | 0.1 | |
| 678/35S:BBLvac-PAO-#9 | TN90 SRC | 1.4 | 0.1292 | 0.2 | |
| 678/35S:BBLvac-PAO-#12 | TN90 SRC | 1.6 | 0.1641 | 0.3 | |
| 678/35S:BBLvac-PAO-#8 | TN90 SRC | 0.9 | 0.2251 | 0.2 | ave. = 0.2 |
| 678/E4:GUS-#5** | TN90 SRC | ND** | 0.1111 | ND | |
| 678/35S:PAO-#6 | TN90 SRC | ND | 0.1474 | ND | |
| 678/35S:BBLa=#7 | TN90 SRC | ND | 0.1091 | ND | |
| 678/35S:BBLvac-PAO-#12 | TN90 SRC | ND | 0.2440 | ND | |

*average value of two independent replications
**the last four leaves are negative controls that were only given buffer during the course of the experiment; ND = not detected.

In a recent manuscript by Lang and Vuarnoz (J. Nat. Prod. 78(1):85-92 (2015)) it was reported that a large proportion of leaf NNK and its alkaloid precursor (speculated to be PON) was found to be tightly associated with the cell wall. Lang and Vuarnoz specifically implicated the lignin fraction of the cell wall as the site of this “matrix-bound NNK” and its precursor. In air-cured burley tobaccos that were assayed, 77% of the leaf NNK was found bound to the extracellular matrix and in flue-cured tobaccos this proportion was 53% (Lang and Vuarnoz, 2015). In both cases, the remaining NNK was soluble, likely representing an intracellular fraction.

PAO enzymes targeted to the cytosol or vacuole would likely be able to metabolize soluble, intracellular pools of PON, but would not be predicted to be able to access or degrade pools of PON localized to the extracellular space (also referred to as the apoplast). Redirecting PAO activity to the apoplast should enable access of the enzyme to the matrix-associated PON fraction, and thus represent an additional means for lowering NNK synthesis beyond that which could be accomplished by targeting PAO to the cytosol and/or vacuole. Targeting proteins to the apoplast can be initiated through the placement of a cleavable signal sequence at the N-terminus of the protein to direct its passage through the endoplasmic reticulum (ER), thus introducing the protein to the secretory pathway. In the absence of additional signals to facilitate either localization to the vacuole or retention within the ER or the Golgi, proteins directed through the secretory pathway are typically transported to the apoplast by a “default pathway” (Hood et al., In A. Altman and P. M. Hasegawa, eds. Plant Biotechnology and Agriculture. Chapter 3. Academic Press, pp. 35-54 (2012)). N-terminal signal sequences are typically 20-30 amino acids long and mediate the co-translational insertion of proteins into the lumen of the ER. Numerous signal sequences have been identified and shown to facilitate extracellular transport of foreign proteins in plant cells. Examples of signal sequences obtained from tobacco genes that have been used to deliver foreign proteins through the secretory system and into the apoplast include those obtained from extensin, PR-S and osmotin (Hood et al., 2012).

Tobacco plants that express PAO enzymes in the apoplast can be generated by engineering constructs in which an ER-directing signal sequence is fused to N-terminus of the PAO enzyme. A protein that has been secreted to the apoplast will typically have free passage throughout the porous cell wall space, if the protein is relatively small and soluble. Tepfer and Taylor (Science 213: 761-763 (1981)) estimated the pores within plant cell walls to be about 4 nm, which should theoretically allow free diffusion of proteins as large as 60 kDa. The predicted molecular mass of the PAO enzyme is 54 kDa, which falls under the proposed 60 kDa threshold. Furthermore, previous publications have reported the targeting of the similar-sized proteins laccase (55 kDa) and cellobiohydrolase (52.5 kDa) to the plant cell wall (Hood et al., Plant Biotech. J. 1: 129-140 (2003); Hood et al. Plant Biotech. J. 5: 709-719 (2007)). Therefore, using an ER tarteting polypeptide fused to PAO or any other PON degrading enzyme should result in a PON enzyme that will freely move throughout the cell wall matrix, and thus will have access to and degrade cell wall-associated PON. Finally, maximal PON degradation and NNK reduction should be accomplished through the generation of transgenic plants containing an apoplast targeted construct, combined with a construct directing PAO to either the cytosol or vacuole (e.g. 35S:PAO and 35S:BBL_{vac}-PAO, respectively). Plants generated in this manner should be capable of reducing both the intracellular soluble pool of PON, as well as the PON pool present in the extracellular matrix, thereby reducing formation of both distinct corresponding pools of NNK as well.

The foregoing is illustrative of the invention, and is not to be construed as limiting thereof. The invention is defined by the following claims, with equivalents of the claims to be included therein.

### Example 5. Analysis of T₂ Transgenic Plants

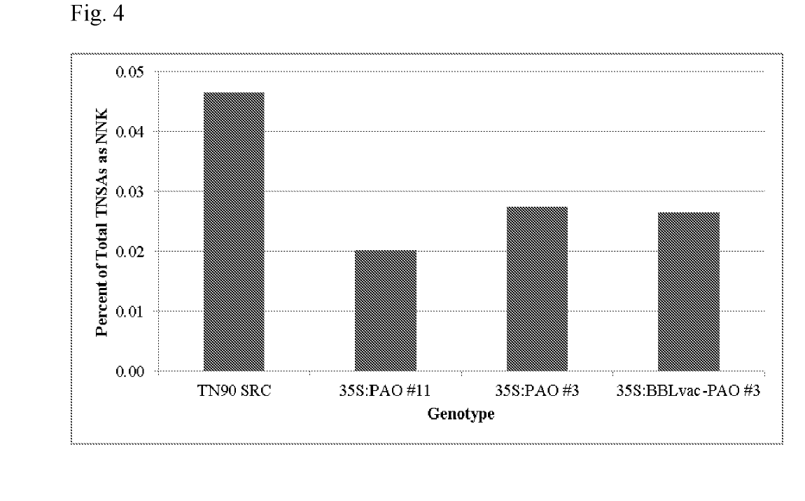

Southern blot analysis was conducted on the T₀ individuals from Table 1 that displayed high d3-PON metabolizing activity. Using the PAO gene as a hybridization probe, we concluded that the PAO transgene was present as a single copy in plants 678/35S:PAO#3, 678/35S:PAO#11, and 678/35S:BBLvac-PAO#3. Each of these plants is within a TN90 SRC burley background. The three single copy PAO T₀ individuals were self-fertilized to produce T₁ generation plants. Several T₁ plants from each line were grown to maturity and numerous T₂ progeny was genotyped to determine the T₁ individuals that were homozygous for the PAO transgene. A total of 30 T₂ plants from seed lots that were fixed for each of the three original T₀ events, as well as TN90 SRC control plants, were grown in the field according to standard industry practice for burley tobaccos. Each plant was stalk-harvested at maturity and air-cured for 10 weeks. At the end of the curing period, the 4^{th} leaf from the top was dried to completion, dried to a fine powder, and analyzed for alkaloid and TSNA content. No significant differences were observed in the alkaloid composition and content between the control and transgenic lines. As shown in FIG. 4, NNK represented about 4.7% of the total TSNA pool in the TN90 SRC control. In contrast, NNK represented only 2.0%, 2.8%, and 2.7% of total TSNAs in lines 35S:PAO#11 (P=0.0084), 35S:PAO#3 (P=0.0550), and 35S:BBLvac-PAO#3(P=0.0537), respectively. These results suggest that the PAO transgene, either with or without a vacuolar localization signal, can reduce the amount of NNK as a percentage of total leaf TSNAs in air-cured burley varieties.

The foregoing is illustrative of the present invention, and is not to be construed as limiting thereof. The invention is defined by the following claims, with equivalents of the claims to be included therein.

APPENDIX
Nucleotide sequences and amino acid sequences

A. Nucleotide sequence of the pseudooxynicotine amine oxidase
(PAO) gene as found in Pseudomonas strain HZN6 (GenBank
accession #JN391188). Start and stop codons are underlined.
SEQ ID NO: 1
ATGGCTAACGATAAGGGTGATATAAGCAAGGACGGTGTATCGCGACGCAAATTTCTTGGCGG
TGCCGTTATTGGTGCTGCTGCTGCTGCAGGCGTTGGCTCTCAGATCCTATCACTGTCCGCTA
CGGCACAGGGGGCGGATAAAGAAAGAGTTGGTCCGCTGCAAAGCAACGTAGATTACGATGCC
GTCGTGATCGGAGGTGGGTTTGCGGGGGTAACGGCTGCAAGGGAGTTGAGCCGATCCGGCTT
GAAAACATTAGTGCTTGAGGGCCGGAGTCGCCTAGGTGGCCGCACGTTTACGTCTAAGCTCG
ATGGTGAGAAGGTGGAGCTTGGAGGTACTTGGGTACACTGGACCCAGCCTAATGTGTGGACT
GAGGTCATGCATTATGGATTAGAAATCGAGGAAACCGTCGGTCTCGCTAGTCCTGAAACTGT
TATTTGGGTTACTGATAATCAGGTGAAGCGAGCGCCGGCGGCAGAGGCGTTCGAAATATTTG
GCGCCGCTTGTACTGAATATTACAAAGAAGCGCACAACATCTACCCACGTCCCTTCGATCCT
TTCTTTGCAAAAAAAGCGCTCCAGGAGATGGATGGGTTGTCAGCTTCTGAGTACTTAAATAA
ACTGTCCCTAACCCGCGAGCAAAAAGACATGATGGATTCATGGCTTAGCGGTAATGGACATA
ACTACCCAGAGACGATCGCTTATAGCGAGATTATGCGCTGGTTTGCACTCAGCAACTTCAAC
ATGCCCACTATGTTCGACTCAATTGCCAGATATAAAATTAAATCAGGTACCGTGAGTCTTCT
GGAGGCCATGGTTGCTGAAAGTGATATGGAAGTTCAGCTTTCAACGCCTGTGCTAAAAGTTA
AGCAAGACAGTCATAGGGTACTTATCACCACTGAAGAGGGCACAATTGCGGCATCGGCAGTT
GTCATGGCAGTGCCTTTAAACACGATGGGTGACGTTGAGTACAGTCCGCGTCTCTCTGATGC
AAAGTCAGAAATTGCCTCCCAAGGCCATGCGGGTAAGGGTGTTAAGGGTTACATTCGTATAA
AGCAAGATGTAGGTAACGTAATGACCTATGCGCCCGCTAGAAACGATGTAACGCCTTTCACT
TCGGTGTTTACGGACCACGTCGGTGAGAACGGTACATTGCTTATCGCATTTTCCGCCGATCC
TAAACTGGTAGACATTAACGATAGCAAAGCGGTCGAAAAGGCCCTGCATCCCCTTCTTCCAG
GCGTGGAGGTAACGTCCAGCTATGGCTACGACTGGAATCTCGATCCCTTTTCTAAGGGCACT
TGGTGCACTTATCGTCCGGGTCAGACAACCCGCTATTTAACCGAGCTGCAGAAGCGCGAAGG
CCGGCTCTTCTTCGCCGGTTCCGACATGGCTAATGGCTGGCGTGGTTTTATAGACGGCGCGA
TAGAGAGCGGTCGCGAGGTCGGGTATCAGGTTGCTAGCTATCTCAAGGGGAAAAATAGCAAT
GCGTGA
B. Nucleotide sequence of the PAO gene used to transform
tobacco cultivars K326 SRC and TN90 SRC. The Pseudomonas HZN6
PAO sequence optimized for expression in tobacco is shown in
black; 5′ and 3′ UTR sequences obtained from the tobacco
CYP82E10 gene are shown in bold; nucleotides engineered to
create restriction sites to facilitate cloning are shown in
italics. Start and stop codons are underlined.
SEQ ID NO: 2
GGATCCAGGGAAGTTGGTGATAGTTTGATTCCCAAGTGCTTTTCTAAAAATCCATACCATGG
CTAATGATAAGGGAGATATAAGTAAAGATGGTGTGAGTAGGAGGAAGTTTCTCGGTGGTGCT
GTGATAGGTGCTGCTGCTGCTGCAGGTGTTGGATCACAAATTTTAAGTCTCTCCGCTACTGC
ACAGGGTGCTGATAAGGAAAGAGTTGGACCACTTCAAAGTAATGTGGATTATGATGCAGTTG
TGATTGGAGGTGGATTTGCTGGTGTGACTGCTGCAAGAGAATTATCTAGATCAGGTCTCAAA
ACACTTGTTTTGGAGGGAAGAAGTAGGTTGGGTGGAAGGACTTTCACATCCAAGTTGGATGG
AGAAAAAGTTGAGTTAGGTGGAACTTGGGTGCATTGGACACAACCAAACGTTTGGACTGAAG
TGATGCACTATGGTCTCGAGATTGAAGAGACAGTTGGACTTGCTTCTCCAGAGACTGTTATA
TGGGTGACAGATAATCAGGTTAAGAGGGCTCCAGCTGCAGAAGCATTTGAGATTTTCGGTGC
TGCATGTACTGAATATTACAAAGAGGCTCATAACATCTATCCAAGGCCTTTCGATCCATTTT
TCGCTAAGAAAGCATTACAAGAGATGGATGGACTCAGTGCTTCCGAGTACCTTAATAAGTTA
TCTCTCACTAGAGAACAGAAAGATATGATGGATTCTTGGCTTTCTGGTAATGGTCACAACTA
TCCAGAAACAATAGCATACTCTGAGATCATGAGATGGTTTGCTCTTTCAAATTTCAACATGC
CTACTATGTTCGATTCAATCGCTAGGTATAAGATAAAAAGTGGTACAGTTTCCCTTTTGGAG
GCTATGGTGGCAGAATCTGATATGGAGGTTCAACTTTCAACTCCAGTTTTGAAGGTGAAACA
GGATTCTCATAGAGTTCTTATCACTACAGAAGAGGGTACTATTGCTGCATCAGCTGTTGTGA
TGGCAGTGCCATTGAATACAATGGGAGATGTTGAATACTCTCCTAGGTTATCAGATGCTAAG
AGTGAGATTGCATCCCAAGGTCACGCTGGAAAGGGAGTTAAAGGATACATCAGAATTAAGCA
GGATGTTGGAAATGTGATGACATACGCTCCAGCAAGGAACGATGTTACTCCTTTTACATCTG
TTTTCACTGATCATGTGGGTGAAAATGGTACTCTTCTCATAGCTTTTAGTGCAGATCCTAAA
CTTGTGGATATCAACGATTCCAAGGCTGTTGAAAAAGCATTGCACCCACTTTTGCCTGGTGT
TGAAGTGACTTCTTCATATGGATACGATTGGAATCTTGATCCATTTTCTAAGGGTACTTGGT
GCACATATAGACCTGGACAAACTACAAGGTACCTTACAGAATTGCAGAAAAGAGAGGGTAGG
CTTTTCTTTGCAGGAAGTGATATGGCTAACGGTTGGAGAGGTTTTATTGATGGTGCTATTGA
ATCCGGTAGGGAGGTTGGTTATCAGGTTGCTTCATATCTCAAGGGAAAGAATAGTAACGCAT
AAAATCTAAGATGTTTTATCTTGGTTGATCATTGTTTAATACTCCTAGATAGATGGGTATTC
ATCTATCTTTTTAAAATTAATTGTCAGTACGAGTGTTTCTGAGCTCAAGCTT
C. Predicted amino acid sequence of PAO
SEQ ID NO: 3
MANDKGDISKDGVSRRKFLGGAVIGAAAAAGVGSQILSLSATAQGADKERVGPLQSNVDYDA
VVIGGGFAGVTAARELSRSGLKTLVLEGRSRLGGRTFTSKLDGEKVELGGTWVHWTQPNVWT
EVMHYGLEIEETVGLASPETVIWVTDNQVKRAPAAEAFEIFGAACTEYYKEAHNIYPRPFDP
FFAKKALQEMDGLSASEYLNKLSLTREQKDMMDSWLSGNGHNYPETIAYSEIMRWFALSNEN
MPTMFDSIARYKIKSGTVSLLEAMVAESDMEVQLSTPVLKVKQDSHRVLITTEEGTIAASAV
VMAVPLNTMGDVEYSPRLSDAKSEIASQGHAGKGVKGYIRIKQDVGNVMTYAPARNDVTPFT
SVFTDHVGENGTLLIAFSADPKLVDINDSKAVEKALHPLLPGVEVISSYGYDWNLDPFSKGT
WCTYRPGQTTRYLTELQKREGRLEFAGSDMANGWRGFIDGAIESGREVGYQVASYLKGKNSN
A
D. Nucleotide sequence of the tobacco BBLa gene (GenBank
Accession #AB604219). Start and stop codons are underlined.
SEQ ID NO: 4
GAAGCAGAAATACATACAACATGTTTCCGCTCATAATTCTGATCAGCTTTTCACTTGCTTCC
TTGTCTGAAACTGCTACTGGAGCTGTTACAAATCTTTCAGCCTGCTTAATCAACCACAATGT
CCATAACTTCTCTATTTACCCCACAAGTAGAAATTACTTTAACTTGCTCCACTTCTCCCTTC
AAAATCTTCGCTTTGCTGCACCTTTCATGCCGAAACCAACCTTCATTATCCTACCAAGCAGT
AAGGAGGAGCTCGTGAGCACCATTTTTTGTTGCAGAAAAGCATCTTATGAAATCAGAGTAAG
GTGCGGCGGACACAGTTACGAAGGAACTTCTTACGTTTCCTTTGACGCTTCTCCATTCGTGA
TCGTTGACTTGATGAAATTAGACGACGTTTCAGTAGATTTGGATTCTGAAACAGCTTGGGCT
CAGGGCGGCGCAACAATTGGCCAAATTTATTATGCCATTGCCAAGGTAAGTGACGTTCATGC
ATTTTCAGCAGGTTCGGGACCAACAGTAGGATCTGGAGGTCATATTTCAGGTGGTGGATTTG
GACTTTTATCTAGAAAATTCGGACTTGCTGCTGATAATGTCGTTGATGCTCTTCTTATTGAT
GCTGATGGACGGTTATTAGACCGAAAAGCCATGGGCGAAGACGTGTTTTGGGCAATCAGAGG
TGGCGGCGGTGGAAATTGGGGCATTGTTTATGCCTGGAAAATTCGATTACTCAAAGTGCCTA
AAATCGTAACAACTTGTATGATCTATAGGCCTGGATCCAAACAATACGTGGCTCAAATACTT
GAGAAATGGCAAATAGTTACTCCAAATTTGGTCGATGATTTTACTCTAGGAGTACTGCTGAG
ACCTGCAGATCTACCCGCGGATATGAAATATGGTAATACTACTCCTATTGAAATATTTCCCC
AATTCAATGCACTTTATTTGGGTCCAAAAACTGAAGTTCTTTCCATATCGAATGAGACATTT
CCGGAGCTAGGCGTTAAGAATGATGAGTGCAAGGAAATGACTTGGGTAGAGTCAGCACTTTT
CTTCTCCGAATTAGCTGACGTTAACGGGAACTCGACTGGTGATATCTCCCGTCTGAAAGAAC
GTTACATGGACGGAAAAGGTTTTTTCAAAGGCAAAACGGACTACGTGAAGAAGCCAGTTTCA
ATGGATGGGATGCTAACATTTCTTGTGGAACTCGAGAAAAACCCGAAGGGATATCTTGTCTT
TGATCCTTATGGCGGAGCCATGGACAAGATTAGTGATCAAGCTATTGCTTTCCCTCATAGAA
AAGGTAACCTTTTCGCGATTCAGTATCTAGCACAGTGGAATGAAGAGGACGATTACATGAGC
GACGTTTACATGGAGTGGATAAGAGGATTTTACAATACAATGACGCCCTTTGTTTCAAGCTC
GCCAAGGGGAGCTTATATCAACTACTTGGATATGGATCTTGGAGTGAATATGGTCGACGACT
ACTTATTGCGAAATGCTAGTAGCAGTAGTCCTTCTTCCTCTGTTGATGCTGTGGAGAGAGCT
AGAGCGTGGGGTGAGATGTATTTCTTGCATAACTATGATAGGTTGGTTAAAGCTAAGACACA
AATTGATCCACTAAATGTTTTTCGACATGAACAGAGTATTCCTCCTATGCTTGGTTCAACGC
AAGAGCACAAGTATAGCAGTGAATGAGATTTAAAATGTACTACCTTGAGAGAGATTCCGTTG
TTAGTTTTCC
E. Predicted amino Acid Sequence of BBLa
SEQ ID NO: 5
MFPLIILISFSLASLSETATGAVTNLSACLINHNVHNESIYPTSRNYFNLLHFSLQNLRFAA
PFMPKPTFIILPSSKEELVSTIFCCRKASYEIRVRCGGHSYEGTSYVSFDASPFVIVDLMKL
DDVSVDLDSETAWAQGGATIGQIYYAIAKVSDVHAFSAGSGPTVGSGGHISGGGFGLLSRKF
GLAADNVVDALLIDADGRLLDRKAMGEDVFWAIRGGGGGNWGIVYAWKIRLLKVPKIVTTCM
IYRPGSKQYVAQILEKWQIVTPNLVDDFTLGVLLRPADLPADMKYGNTTPIEIFPQFNALYL
GPKTEVLSISNETFPELGVKNDECKEMTWVESALFFSELADVNGNSTGDISRLKERYMDGKG
FFKGKTDYVKKPVSMDGMLTFLVELEKNPKGYLVFDPYGGAMDKISDQATAFPHRKGNLFAI
QYLAQWNEEDDYMSDVYMEWIRGEYNTMTPFVSSSPRGAYINYLDMDLGVNMVDDYLLRNAS
SSSPSSSVDAVERARANGEMYFLHNYDRLVKAKTQIDPLNVERHEQSIPPMLGSTQEHKYSS
E
F. Nucleotide sequence of the BBL_{vac}-PAO fusion construct.
Black, italized sequences correspond to the BBLa 5′ flanking
sequence and first 50 codons that contain a vacuolar
localization signal; black, standard font sequences represent
the optimized Pseudomonas HZN6 PAO sequence; 3′ UTR sequence
obtained from the tobacco CYP82E10 gene is shown in bold;
nucleotides engineered to create restriction sites to
facilitate cloning are shown in lowercase. Start and stop
codons are underlined.
SEQ ID NO: 6
ggatccGAAGCAGAAATACATACAACATGTTTCCGCTCATAATTCTGATCAGCTTTTCACTT
GCTTCCTTGTCTGAAACTGCTACTGGAGCTGTTACAAATCTTTCAGCCTGCTTAATCAACCA
CAATGTCCATAACTTCTCTATTTACCCCACAAGTAGAAATTACTTTAACTTGgccATGGCTA
ATGATAAGGGAGATATAAGTAAAGATGGTGTGAGTAGGAGGAAGTTTCTCGGTGGTGCTGTG
ATAGGTGCTGCTGCTGCTGCAGGTGTTGGATCACAAATTTTAAGTCTCTCCGCTACTGCACA
GGGTGCTGATAAGGAAAGAGTTGGACCACTTCAAAGTAATGTGGATTATGATGCAGTTGTGA
TTGGAGGTGGATTTGCTGGTGTGACTGCTGCAAGAGAATTATCTAGATCAGGTCTCAAAACA
CTTGTTTTGGAGGGAAGAAGTAGGTTGGGTGGAAGGACTTTCACATCCAAGTTGGATGGAGA
AAAAGTTGAGTTAGGTGGAACTTGGGTGCATTGGACACAACCAAACGTTTGGACTGAAGTGA
TGCACTATGGTCTCGAGATTGAAGAGACAGTTGGACTTGCTTCTCCAGAGACTGTTATATGG
GTGACAGATAATCAGGTTAAGAGGGCTCCAGCTGCAGAAGCATTTGAGATTTTCGGTGCTGC
ATGTACTGAATATTACAAAGAGGCTCATAACATCTATCCAAGGCCTTTCGATCCATTTTTCG
CTAAGAAAGCATTACAAGAGATGGATGGACTCAGTGCTTCCGAGTACCTTAATAAGTTATCT
CTCACTAGAGAACAGAAAGATATGATGGATTCTTGGCTTTCTGGTAATGGTCACAACTATCC
AGAAACAATAGCATACTCTGAGATCATGAGATGGTTTGCTCTTTCAAATTTCAACATGCCTA
CTATGTTCGATTCAATCGCTAGGTATAAGATAAAAAGTGGTACAGTTTCCCTTTTGGAGGCT
ATGGTGGCAGAATCTGATATGGAGGTTCAACTTTCAACTCCAGTTTTGAAGGTGAAACAGGA
TTCTCATAGAGTTCTTATCACTACAGAAGAGGGTACTATTGCTGCATCAGCTGTTGTGATGG
CAGTGCCATTGAATACAATGGGAGATGTTGAATACTCTCCTAGGTTATCAGATGCTAAGAGT
GAGATTGCATCCCAAGGTCACGCTGGAAAGGGAGTTAAAGGATACATCAGAATTAAGCAGGA
TGTTGGAAATGTGATGACATACGCTCCAGCAAGGAACGATGTTACTCCTTTTACATCTGTTT
TCACTGATCATGTGGGTGAAAATGGTACTCTTCTCATAGCTTTTAGTGCAGATCCTAAACTT
GTGGATATCAACGATTCCAAGGCTGTTGAAAAAGCATTGCACCCACTTTTGCCTGGTGTTGA
AGTGACTTCTTCATATGGATACGATTGGAATCTTGATCCATTTTCTAAGGGTACTTGGTGCA
CATATAGACCTGGACAAACTACAAGGTACCTTACAGAATTGCAGAAAAGAGAGGGTAGGCTT
TTCTTTGCAGGAAGTGATATGGCTAACGGTTGGAGAGGTTTTATTGATGGTGCTATTGAATC
CGGTAGGGAGGTTGGTTATCAGGTTGCTTCATATCTCAAGGGAAAGAATAGTAACGCATAAA
ATCTAAGATGTTTTATCTTGGTTGATCATTGTTTAATACTCCTAGATAGATGGGTATTCATC
TATCTTTTTAAAATTAATTGTCAGTACGAGTGTTTCTgagctcaagctt
G. Predicted amino acid sequence of BBL_{vac}-PAO fusion protein.
Amino acids obtained from BBLa are the first 50 amino acids
shown in lighter font; residues shown in black correspond to
PAO; the bold, underlined alanine residue was generated as a
consequence of creating the fusion construct.
SEQ ID NO: 7
MFPLTILISFSLASLSETATGAVTNLSACLINHNVHNESIYPTSRNYFNLAMANDKGDISKD
GVSRRKFLGGAVIGAAAAAGVGSQILSLSATAQGADKERVGPLQSNVDYDTVVIGGGFAGVT
AARELSRSGLKTLVLEGRSRLGGRTFTSKLDGEKVELGGTWVHWTQPNVWTEVMHYGLEIEE
TVGLASPETVIWVTDNQVKRAPAAEAFEIFGAACTEYYKEAHNIYPRPFDPFFAKKALQEMD
GLSASEYLNKLSLTREQKDMMDSWLSGNGHNYPETIAYSEIMRWFALSNFNMPTMFDSIARY
KIKSGTVSLLEAMVAESDMEVQLSTPVLKVKQDSHRVLITTEEGTIAASAVVMAVPLNTMGD
VEYSPRLSDAKSEIASQGHAGKGVKGYIRIKQDVGNVMTYAPARNDVTPFTSVFTDHVGENG
TLLIAFSADPKLVDINDSKAVEKALHPLLPGVEVTSSYGYDWNLDPFSKGTWCTYRPGQTTR
YLTELQKREGRLFFAGSDMANGWRGFIDGAIESGREVGYQVASYLKGKNSNA
H. Vacuolar Targeting Sequence of BBLa
SEQ ID NO: 8
GAAGCAGAAATACATACAACATGTTTCCGCTCATAATTCTGATCAGCTTTTCACTTGCTTCC
TTGTCTGAAACTGCTACTGGAGCTGTTACAAATCTTTCAGCCTGCTTAATCAACCACAATGT
CCATAACTTCTCTATTTACCCCACAAGTAGAAATTACTTTAACTTG
I. Nucleotide sequence of the Putative PAO Gene from
Pseudomonas putida S16.
SEQ ID NO: 9
ATGACAAAAGATGGTGATGAAGGCAGCAAAAGCGGAGTATCACGCCGAAAGTTCCTTGGTAG
CGCCGCGGTCGGAGTGGCAACAGCGGGCATAGCCTCGCAGCTTCTGACTCTGTCGGCGCCCG
CGGAAGCGGCGGTGAAGACCAATGTTGGTCCATCACGCGCAGGCGTGGGTTATGACGTTATT
GTAATCGGTGGAGGCTTCGCGGGTGTTACTGCGGCGCGAGAAGCAAGCCGTTCTGGCTTGAA
AACTCTAATTCTTGAAGGTAGAAGTCGGTTGGGCGGCCGAACTTTTACGTCTAAGCTTCAGA
ATCAAAAAGTTGAGTTGGGAGGTACCTGGGTACATTGGACCCAGCCGAATGTTTGGACTGAG
ATTATGCACTATGGCCTAGAGGTGGAGGAGACGGTCGGTCTTGCGAATCCAGAGACCGTCAT
TTGGGTAACAGAGGATAATGTCAAAAGAGCACCTGCAGCAGAGGCGTTTGAAATTTTTGGCT
CTGCCTGTAACGAATACTACAAAGAAGCACGGAATATTTATCCGCGCCCGTTTGAACCATTT
TTTGAGCGAAAGAAGCTGCAGCATGTTGATGGACTTTCCGCGGCAGATTATCTTGAAAAATT
GCCTTTGACCAGGGAGCAAAAGGATATGATGGATTCCTGGCTCAGTGGAAATGGACATAATT
ATCCGGAAACTATTGCATATAGCGAGATTATGCGTTGGTTTGCACTCAGTAACTTTAATATG
CCCACAATGTTCGATTCCATTGCGCGGTACAAAATTAAAACCGGTACGCATAGTCTTTTAGA
GGCCATAATGGCGGATGGTAACTCGGAAGTTAAACTTTCGACGCCAGTGACTAAGGTTAATC
AAGATAAAGATAAAGTAACAGTTACCACTGAAGACGGTGTCTTCACGGCATCAGCGGTAATT
GTAGCCGTCCCTATCAACACTTTGCATGATATTGAGTACTCGCCGAAGTTGTCGGCAGCTAA
AGTGGATATGGGATCGCAACGGCATGCTGGTGCTGGTGTAAAGGGCTATATTCGCGTAAAAC
AAAATGTCGGCAATGTAATGACATATGCTCCTGCCCGGAATAAGCTCACACCATTTACCTCA
GTATTCACAGATCACGTGGACGAGAGCGGTACGTTACTCATTGCATTTTCAGCCGACCCTAA
GTTGATTGATATCAATGATATCAAAGCCGTCGAAAAGGCTTTGCAACCACTTTTGCCAGGCG
TCGAAGTAACTGCTAGTTATGGCTACGACTGGAACCTCGATCCCTTTTCTAAGGGCACTTGG
TGCACTTACCGTCCTAACCAGACGACTCGATACTTGACTGAGCTGCAGAAGCGCGAAGGACG
GCTATTTTTTGCAGGCTCGGACATGGCCAATGGTTGGCGTGGATTCATTGATGGAGCAATCG
AGAACGGTAGAGAGGTAGGGCATCAAGTCGCTACGTATCTAAAAAGAGAAAATGACAATGCG
TGA
J. Predicted protein sequence of the Putative PAO Gene from
Pseudomonas putida S16:
SEQ ID NO: 10
mtkdgdegsksgvsrrkflgsaavgvatagiasqlltlsapaeaavktnvgpsragvgydvi
vigggfagvtaareasrsglktlilegrsrlggrtftsklqnqkvelggtwvhwtqpnvwte
imhygleveetvglanpetviwvtednvkrapaaeafeifgsacneyykearniyprpfepf
ferkklqhvdglsaadyleklpltreqkdmmdswlsgnghnypetiayseimrwfalsnfnm
ptmfdsiarykiktgthslleaimadgnsevklstpvtkvngdkdkvtvttedgyftasavi
vavpintlhdieyspklsaakvdmgsgrhagagvkgyirvkqnvgnvmtyaparnkltpfts
vftdhvdesgtlliafsadpklidindikavekalqpllpgvevtasygydwnldpfskgtw
ctyrpngttryltelqkregrlffagsdmangwrgfidgaiengrevghqvatylkrendna

That which is claimed is:

1. A tobacco plant, plant part, and/or plant cell comprising one or more heterologous nucleic acid molecules comprising a nucleotide sequence encoding a pseudooxynicotine (PON) degrading enzyme.

2. The tobacco plant, plant part and/or plant cell of claim 1, wherein the PON degrading enzyme is from a fungal or bacterial genera known to degrade nicotine.

3. The tobacco plant, plant part and/or plant cell of claim 1 or claim 2, wherein the PON degrading enzyme is a pseudooxynicotine amine oxidase (PAO).

4. The tobacco plant, plant part and/or plant cell of claim 3, wherein the PAO is from Pseudomonas strain HZN6 or Pseudomonas putida S16.

5. The tobacco plant, plant part, and/or plant cell of claim 3 or claim 4, wherein the nucleotide sequence encoding PAO comprises the nucleotide sequence of SEQ ID NO:1 or SEQ ID NO:2.

6. The tobacco plant, plant part, and/or plant cell of any one of claims 3 to 5, wherein the nucleotide sequence encoding PAO encodes a polypeptide comprising the amino acid sequence of SEQ ID NO:3.

7. The tobacco plant, plant part and/or plant cell of any one of claims 1 to 6, wherein the PON degrading enzyme encoded by the nucleotide sequence is fused to a vacuolar targeting sequence.

8. The tobacco plant, plant part and/or plant cell of any one of claims 1 to 6, wherein the PON degrading enzyme encoded by the nucleotide sequence is fused to an endoplasmic reticulum (ER) targeting signal sequence.

9. The tobacco plant, plant part and/or plant cell of any one of claims 1 to 6, comprising at least two heterologous nucleic acid molecules comprising a nucleotide sequence encoding a PON degrading enzyme, wherein at least one of the at least two heterologous nucleic acid molecules comprises a nucleotide sequence encoding a PON degrading enzyme fused to a vacuolar targeting sequence and at least one of the at least two heterologous nucleic acid molecules comprises a nucleotide sequence encoding a PON degrading enzyme fused to a ER targeting signal sequence.

10. The tobacco plant, plant part and/or plant cell of claim 7 or claim 9, wherein the vacuolar targeting sequence is encoded by the nucleotide sequence of SEQ ID NO:8.

11. The tobacco plant, plant part and/or plant cell any one of claim 7, 9 or 10, wherein the PON degrading enzyme fused to a vacuolar targeting sequence is encoded by the nucleotide sequence of SEQ ID NO:6.

12. The tobacco plant, plant part and/or plant cell of any one of claim 7, 9 or 11, wherein the PON degrading enzyme fused to a vacuolar targeting sequence comprises the amino acid sequence of SEQ ID NO:7.

13. The tobacco plant, plant part and/or plant cell of any one of claims 1 to 12, wherein the nucleic acid molecule further comprises one or more regulatory sequences.

14. A progeny plant or seed produced from the tobacco plant, plant part or plant cell of any one of claims 1-13, wherein said progeny plant or seed comprises the one or more heterologous nucleic acid molecules comprising a nucleotide sequence encoding a PON degrading enzyme.

15. A seed from the progeny plant of claim 14, wherein said seed comprises the one or more heterologous nucleic acid molecules comprising a nucleotide sequence encoding a PON degrading enzyme.

16. A tobacco plant produced by the seed of claim 14 or claim 15, wherein said plant comprises the one or more heterologous nucleic acid molecules comprising a nucleotide sequence encoding a PON degrading enzyme.

17. A plant crop comprising a plurality of the tobacco plants of any one of claims 1-14 or 16 planted together in an agricultural field.

18. A tobacco product produced from the tobacco plant, plant part and/or plant cell of any one of claims 1 to 13, from the progeny plant of claim 14, from the seed of claim 14 or 15, from the plant of claim 16, and/or from the crop of claim 17.

19. The tobacco product of claim 18, wherein the tobacco product is leaf tobacco, shredded tobacco, cut tobacco, ground tobacco, powder tobacco, tobacco extract, nicotine extract, smokeless tobacco, moist or dry snuff, kretek, pipe tobacco, cigar tobacco, cigarillo tobacco, cigarette tobacco, chewing tobacco, bidis, bits, and tobacco-containing gum, lozenges, patches, electronic cigarettes, or any combination thereof.

20. The tobacco product of claim 18, wherein the tobacco product is a cigarette, cigarillo, a non-ventilated recess filter cigarette, a vented recess filter cigarette, a cigar, snuff, chewing tobacco or any combination thereof.

21. The tobacco product of any one of claims 18 to 20, wherein the product has a reduced amount of PON and/or NNK.

22. A method of reducing pseudooxynicotine (PON) and/or 4-(methyl nitrosoamino)-1-(3-pyridyl)-1-butanone (NNK) in a tobacco plant, plant part and/or plant cell, comprising: introducing into a tobacco plant, plant part and/or plant cell one or more heterologous nucleic acid molecules comprising a nucleotide sequence encoding a PON degrading enzyme, thereby reducing the PON and/or NNK content in the transgenic tobacco plant and/or plant part.

23. A method of producing a plant, plant part and/or plant cell having reduced pseudooxynicotine (PON) and/or 4-(methyl nitrosoamino)-1-(3-pyridyl)-1-butanone (NNK) content, comprising: introducing into a tobacco plant, plant part and/or plant cell one or more heterologous nucleic acid molecules comprising a nucleotide sequence encoding a PON degrading enzyme, thereby producing a tobacco plant, plant part and/or plant cell having reduced PON and/or NNK content.

24. A method of producing a tobacco product having reduced PON and/or NNK content, the method comprising: producing a tobacco product from the tobacco plant, plant part and/or plant cell of any one of claims 1 to 14, or 16, or the crop of claim 17, wherein the tobacco product has reduced PON and/or NNK content.

25. A method of producing a tobacco product having reduced having reduced pseudooxynicotine (PON) and/or 4-(methyl nitrosoamino)-1-(3-pyridyl)-1-butanone (NNK) content, comprising: introducing into a tobacco plant, plant part and/or plant cell one or more heterologous nucleic acid molecules comprising a nucleotide sequence encoding a PON degrading enzyme, thereby producing a transgenic tobacco plant, plant part and/or plant cell having reduced PON and/or NNK content; and producing a tobacco product from said transgenic tobacco plant, plant part and/or plant cell, wherein the tobacco product has reduced PON and/or NNK content.

26. The method of any one of claims 22 to 25, wherein the nucleotide sequence encoding a PON degrading enzyme is from a fungal and/or bacterial genera known to degrade nicotine.

27. The method of any one of claims 22 to 26, wherein the PON degrading enzyme is a PAO.

28. The method of claim 27, wherein the PAO is from Pseudomonas strain HZN6 or Pseudomonas putida S16.

29. The method of claim 26 or claim 27, wherein the nucleotide sequence encoding PAO comprises the nucleotide sequence of SEQ ID NO:1 or SEQ ID NO:2.

30. The method of any one of claims 27 to 29, wherein the nucleotide sequence encoding PAO encodes a polypeptide comprising the amino acid sequence of SEQ ID NO:3.

31. The method of any one of claims 22 to 30, wherein the PON degrading enzyme encoded by the nucleotide sequence is fused to a vacuolar targeting sequence.

32. The method of any one of claims 22 to 30, wherein the PON degrading enzyme encoded by the nucleotide sequence encoding is fused to an endoplasmic reticulum targeting signal sequence.

33. The method of any one of claims 22 to 30, comprising at least two heterologous nucleic acid molecules comprising a nucleotide sequence encoding a PON degrading enzyme, wherein at least one of the at least two heterologous nucleic acid molecules comprises a nucleotide sequence encoding a PON degrading enzyme fused to a vacuolar targeting sequence and at least one of the at least two heterologous nucleic acid molecules comprises a nucleotide sequence encoding a PON degrading enzyme fused to a ER targeting signal sequence.

34. The method of claim 31 or claim 33, wherein the vacuolar targeting sequence is encoded by the nucleotide sequence of SEQ ID NO:8.

35. The method of any one of claim 31, 33 or 34, wherein the PON degrading enzyme fused to a vacuolar targeting sequence is encoded by the nucleotide sequence of SEQ ID NO:6.

36. The method of any one of claim 31, or 33 to 35, wherein the PON degrading enzyme fused to a vacuolar targeting sequence comprises the amino acid sequence of SEQ ID NO:7.

37. The method of any one of claims 22 to 36, wherein the nucleic acid molecule further comprises one or more regulatory sequences.

38. A tobacco plant, plant part, and/or plant cell produced by the method of any one of claims 22 to 37.

39. A progeny plant or seed produced from the tobacco plant, plant part or plant cell of claim 38, wherein said progeny plant or seed comprises the one or more heterologous nucleic acid molecules cule comprising a nucleotide sequence encoding a PON degrading enzyme.

40. A seed from the progeny plant of claim 39, wherein said seed comprises the one or more heterologous nucleic acid molecules comprising a nucleotide sequence encoding a PON degrading enzyme.

41. A tobacco plant from the seed of claim 39 or claim 40, wherein said plant comprises the one or more heterologous nucleic acid molecules comprising a nucleotide sequence encoding a PON degrading enzyme.

42. A plant crop comprising a plurality of transgenic tobacco plants of any one of claim 38, 39 or 41 planted together in an agricultural field.

43. A tobacco product produced from the method of claim 24.

44. A tobacco product produced from the tobacco plant, plant part and/or plant cell of claim 38, from the progeny plant of claim 39, from the seed of claim 39 or 40, from the tobacco plant of claim 40, and/or from the crop of claim 41

45. The tobacco product of claim 43 or claim 44, wherein the tobacco product is leaf tobacco, shredded tobacco, cut tobacco, ground tobacco, powder tobacco, tobacco extract, nicotine extract, smokeless tobacco, moist or dry snuff, kretek, pipe tobacco, cigar tobacco, cigarillo tobacco, cigarette tobacco, chewing tobacco, bidis, bits, and tobacco-containing gum, lozenges, electronic cigarettes, or any combination thereof.

46. The tobacco product of claim 43 or claim 44, wherein the tobacco product is a cigarillo, a cigarette, a non-ventilated recess filter cigarette, a vented recess filter cigarette, a cigar, snuff, chewing tobacco or any combination thereof.

47. The tobacco product of any one of claims 43 to 46, wherein the product has a reduced amount of PON and/or NNK.
